(19) United States
(12) Patent Application Publication
(10) Pub. No.: US 2025/0198015 A1
(43) Pub. Date: Jun. 19, 2025

(54) METHODS AND SYSTEMS FOR THE ELECTROCHEMICAL CONVERSION OF CARBON DIOXIDE AND STEAM TO SYNGAS

(71) Applicant: Chevron U.S.A. Inc., San Ramon, CA (US)

(72) Inventors: Zihan Huang, Emeryville, CA (US); Lin Li, San Ramon, CA (US)

(21) Appl. No.: 18/538,320

(22) Filed: Dec. 13, 2023

(51) Int. Cl. C25B 1/23; C10G 2/00; C25B 13/05; C25B 15/027; C25B 15/08

(52) U.S. Cl. CPC C25B 1/23; C10G 2/40; C25B 13/05; C25B 15/027; C25B 15/081; C10G 2400/02; C10G 2400/04; C10G 2400/08

(57) ABSTRACT

A method includes heating a carbon dioxide feed stream in a first heat exchanger using a first cathode effluent from a cathode of an electrolyzer to generate a heated carbon dioxide effluent, heating a first steam feed stream in a second heat exchanger using a second cathode effluent from the cathode of the electrolyzer to generate a first heated steam effluent, heating a second steam feed stream in a third heat exchanger using an anode effluent from an anode of the electrolyzer to generate a second heated steam effluent, combusting, in a combustion unit, a tail gas stream to transfer heat from the combusting to the heated carbon dioxide effluent, the first heated steam effluent and the second heated steam effluent to generate a heated carbon dioxide and steam stream effluent, and passing the heated carbon dioxide and steam stream effluent to the cathode of the electrolyzer.

### BACKGROUND

Many energy production processes release carbon dioxide. In order to reduce the climate-damaging effects of carbon dioxide in the atmosphere, the carbon dioxide can be converted into other substances. However, this is made more difficult by the fact that carbon dioxide is stable and not very reactive. In addition, the carbon in the carbon dioxide molecule is in its highest oxidation state. Thus, carbon dioxide can no longer be used to generate energy by means of incineration.

However, carbon dioxide can be reduced to, for example, carbon monoxide by supplying energy. The carbon in carbon monoxide is in a lower oxidation state than in carbon dioxide, and, therefore, can be used for more applications than carbon dioxide. Accordingly, converting carbon dioxide into a chemical such as carbon monoxide not only reduces the amount of climate-damaging carbon dioxide, but also produces a valuable chemical raw material such as carbon monoxide.

For example, carbon monoxide can be combined with hydrogen to form a syngas containing the elements carbon, oxygen and hydrogen, which are necessary for the production of important organic chemicals. Syngas is suitable for many petrochemical processes such as, for example, the production of synthetic fuel, natural gas, methanol, formaldehyde, etc.

Producing a chemical using energy is also known as “Power-to-X” because energy (“power”) can be used to obtain a chemical (“X”). By using climate-damaging carbon dioxide as a starting material, this concept can contribute to reducing global warming.

### SUMMARY

In accordance with an illustrative embodiment, a method comprises:
heating a carbon dioxide feed stream in a first heat exchanger using a first heated cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
heating a first steam feed stream having a temperature of about 250° C. to about 350° C. in a second heat exchanger using a second heated cathode effluent from the cathode of the electrolyzer as a heat transfer medium to generate a first heated steam effluent having a temperature of about 550° C. to about 650° C.,
heating a second steam feed stream having a temperature of about 250° C. to about 350° C. in a third heat exchanger using an anode effluent from the anode of the electrolyzer as a heat transfer medium to generate a second heated steam effluent having a temperature of about 550° C. to about 650° C.,
combusting, in a combustion unit, a tail gas stream to transfer heat from the combusting to the heated carbon dioxide effluent, the first heated steam effluent and the second heated steam effluent to generate a heated carbon dioxide and steam stream effluent having a temperature of about 700° C. to about 950° C., and
passing the heated carbon dioxide and steam stream effluent to the cathode of the electrolyzer.

In accordance with another illustrative embodiment, a method comprises:
heating a water feed stream in a first heat exchanger using a reactor synthesis effluent including tail gas from a reactor unit as a heat transfer medium to generate a heated water effluent having a temperature of about 50° C. to about 150° C. and a cooled reactor synthesis effluent including the tail gas,
separating the tail gas from the cooled reactor synthesis effluent to generate a first tail gas stream and a second tail gas stream,
introducing the first tail gas stream, a cooled cathode effluent and the heated water effluent to the reactor unit,
performing an exothermic reaction comprising the first tail gas stream and the cooled cathode effluent in the reactor unit thereby transferring heat from the exothermic reaction to the heated water effluent to generate a steam feed stream having a temperature of about 250° C. to about 350° C.,
splitting the steam feed stream to a first steam feed stream and a second steam feed stream,
heating a carbon dioxide feed stream in a second heat exchanger using a first heated syngas effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
heating the first steam feed stream in a third heat exchanger using a second heated syngas effluent from the cathode of the electrolyzer as a heat transfer medium to generate a first heated steam effluent having a temperature of about 550° C. to about 650° C.,
heating the second steam feed stream in a fourth heat exchanger using an anode effluent from the anode of the electrolyzer as a heat transfer medium to generate a second heated steam effluent having a temperature of about 550° C. to about 650° C.,
combusting, in a combustion unit, the second tail gas stream to transfer heat from the combusting to heat the heated carbon dioxide effluent, the first heated steam effluent and the second heated steam effluent and generate a heated carbon dioxide and steam stream effluent having a temperature of about 700° C. to about 950° C., and
passing the heated carbon dioxide and steam stream effluent to the cathode of the electrolyzer.

In accordance with yet another illustrative embodiment, a system comprises:
a first heat exchanger configured to heat a carbon dioxide feed stream using a first heated cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
a second heat exchanger configured to heat a first steam feed stream having a temperature of about 250° C. to about 350° C. using a second heated cathode effluent from the cathode of the electrolyzer as a heat transfer medium to generate a first heated steam effluent having a temperature of about 550° C. to about 650° C.,
a third heat exchanger configured to heat a second steam feed stream having a temperature of about 250° C. to about 350° C. using an anode effluent from the anode of the electrolyzer as a heat transfer medium to generate a second heated steam effluent having a temperature of about 550° C. to about 650° C., and
a combustion unit configured to combust a tail gas feed stream to transfer heat from the combusting to the heated carbon dioxide effluent, the first heated steam effluent and the second heated steam effluent to generate a heated carbon dioxide and steam stream effluent having a temperature of about 700° C. to about 950° C. for sending to the cathode of the electrolyzer.

### BRIEF DESCRIPTION OF THE DRAWINGS

In combination with the accompanying drawing and with reference to the following detailed description, the features, advantages, and other aspects of the implementations of the present disclosure will become more apparent, and several implementations of the present disclosure are illustrated herein by way of example but not limitation. In the accompanying drawings:

FIG. 1 is a flow chart illustrating a method utilizing the starting feed streams with heat integration design including a combustion unit for the electrochemical conversion of carbon dioxide and steam to produce syngas for production of methanol and dimethyl ether, according to an illustrative embodiment.

FIG. 2A illustrates a process flow diagram scheme with heat integration design including a combustion unit for the electrochemical conversion of carbon dioxide and steam to produce syngas for production of methanol and dimethyl ether, according to an illustrative embodiment.

FIG. 2B illustrates a process flow diagram scheme with heat integration design including a combustion unit for the electrochemical conversion of carbon dioxide and steam to produce syngas for production of methanol and dimethyl ether, according to an alternative illustrative embodiment.

FIG. 3A illustrates a process flow diagram scheme with heat integration design including a combustion unit for the electrochemical conversion of carbon dioxide and steam to produce syngas for production of Fischer-Tropsch products, according to an illustrative embodiment.

FIG. 3B illustrates a process flow diagram scheme with heat integration design including a combustion unit for the electrochemical conversion of carbon dioxide and steam to produce syngas for production of Fischer-Tropsch products, according to an alternative illustrative embodiment.

FIG. 4 is a flow chart illustrating a method utilizing the starting feed stream with heat integration design without a combustion unit for the electrochemical conversion of carbon dioxide and steam to produce syngas for production of methanol and dimethyl ether, according to an alternative illustrative embodiment.

FIG. 5A illustrates a process flow diagram scheme with heat integration design without a combustion unit for the electrochemical conversion of carbon dioxide and steam to produce syngas for production of methanol and dimethyl ether, according to an illustrative embodiment.

FIG. 5B illustrates a process flow diagram scheme with heat integration design without a combustion unit for the electrochemical conversion of carbon dioxide and steam to produce syngas for production of methanol and dimethyl ether, according to an alternative illustrative embodiment.

FIG. 6A illustrates a process flow diagram scheme with heat integration design without a combustion unit for the electrochemical conversion of carbon dioxide and steam to produce syngas for production of Fischer-Tropsch products, according to an illustrative embodiment.

FIG. 6B illustrates a process flow diagram scheme with heat integration design without a combustion unit for the electrochemical conversion of carbon dioxide and steam to produce syngas for production of Fischer-Tropsch products, according to an alternative illustrative embodiment.

### DETAILED DESCRIPTION

Various illustrative embodiments described herein are directed to methods and systems for the electrochemical conversion of carbon dioxide into syngas for the production of chemical products and/or fuels. “Power-to-gas”, “power-to-liquid”, and “power-to-fuel” (referred to as “Power-to-X” processes) represent promising approaches for bringing about a future conversion from fossil energy sources to an energy infrastructure which is based mainly on renewable energy sources (RES), for example wind power, solar power, geothermal energy or water power. Electricity-based or synthetic fuels are becoming ever more important, particularly in the transport sector or in industry. Such fuels, for example methane, methanol or derivatives or downstream products such as kerosene, gasoline, diesel, or other hydrocarbon-based products are produced, in particular, by synthesis from hydrogen and carbon dioxide.

Power-to-X processes utilize electrical energy to convert carbon dioxide into carbon-neutral fuels or chemicals. For example, it is possible to convert carbon dioxide and water directly into multi-carbon species as desired fuels or chemicals. However, such a direct carbon conversion route for fuel synthesis is still at the early stage of development and faces significant technical challenges. On the other hand, the indirect route involves carbon dioxide and water being first converted into hydrogen and carbon monoxide by electrolysis, which can be further transformed into desired products in a downstream synthesis reaction process, e.g., an electrolyzer by way of co-electrolysis can convert water and carbon dioxide to syngas, and the produced syngas can be further reacted to make such chemical products as methanol. However, for the indirect route, the electrolysis of water and carbon dioxide requires a large amount of energy. Accordingly, improving the energy efficiency of the Power-to-X process is critical to make the technology more economically feasible.

With energy available from renewable sources, the energy requirement of power-to-fuel process may still be high for large scale applications, therefore it is critical for the system to achieve high energy efficiency for practical application. A critical optimization area to help achieve high energy efficiency of the process is to provide enough thermal energy of material streams to an electrolyzer, e.g., heat each cold feed inlet of water, carbon dioxide and air, for operation of the electrolyzer using the available heat sources within the process and system via efficient heat integration design by reducing the consumption of external energy input.

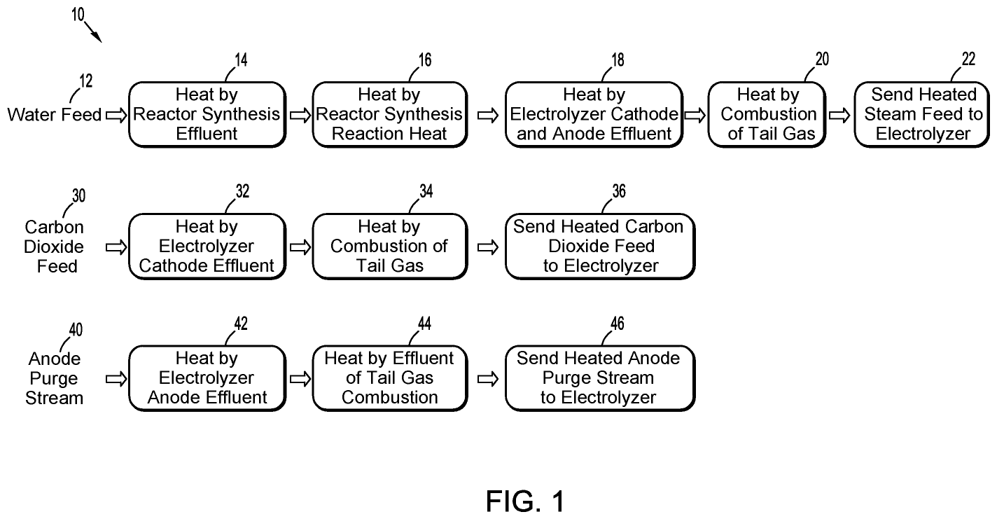

The illustrative embodiments disclosed herein of disclosed herein of FIGS. 1-6B therefore utilize heat integration design and the specific heat integration design of recovering heat from various heat sources of the method and system disclosed herein (i.e., reaction heat of fuel synthesis, heat recovered from fuel cooling, hot cathode effluent, combustion heat of tail gas, etc.) to heat the material streams to the electrolyzer to supply all thermal energy required to minimize the external electrical energy input (or maximize the overall process efficiency).

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and methods are described in terms of “comprising” various components or steps, the systems and methods can also “consist essentially of” or “consist of” the various components or steps, unless stated otherwise.

The terms “a,” “an,” and “the” are intended to include plural alternatives, e.g., at least one. The terms “including”, “with”, and “having”, as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “electrode” is meant, in the sense of the present disclosure, an electronic conductor capable of capturing or releasing electrons. An oxidation reaction occurs at the anode, whereas a reduction reaction occurs at the cathode.

The term “electrolysis” is a technique that uses direct electric current to drive an otherwise non-spontaneous chemical reaction. For example, the electrolysis of water is the process of using electricity to electrochemically decompose water into oxygen and hydrogen. The term “electrolyzer,” also called an “electrolysis device,” refers to a unit where this chemical reaction may take place.

The non-limiting illustrative embodiments described herein overcome the drawbacks discussed above by providing methods and systems for the electrochemical conversion of carbon dioxide to chemicals and/or fuels. For example, the non-limiting illustrative embodiments described herein include a system-level process design with optimized heat integration of an electrolyzer coupled with a downstream synthesis reaction process, from which the overall system efficiency is enhanced by leveraging the available heat sources in the process with relatively minimal reliance on combustion heat. Accordingly, the non-limiting illustrative embodiments described herein are able to maximize the utilization of the available heat sources to generate steam and heat up the feed streams being sent to the electrolyzer, thereby reducing the electrolyzer electricity consumption while improving energy efficiency and reducing electricity cost.

The illustrative embodiments of the present disclosure will be specifically described below with reference to the accompanying drawings. For the purpose of clarity, some steps leading up to the production of the electrochemical conversion of carbon dioxide to chemicals and/or fuels as illustrated in FIGS. 1-6B are omitted. In other words, one or more well-known processing steps which are not illustrated but are well-known to those of ordinary skill in the art have not been included in the figures. This is not intended to be interpreted as a limitation of any particular embodiment, or illustration, or scope of the claims.

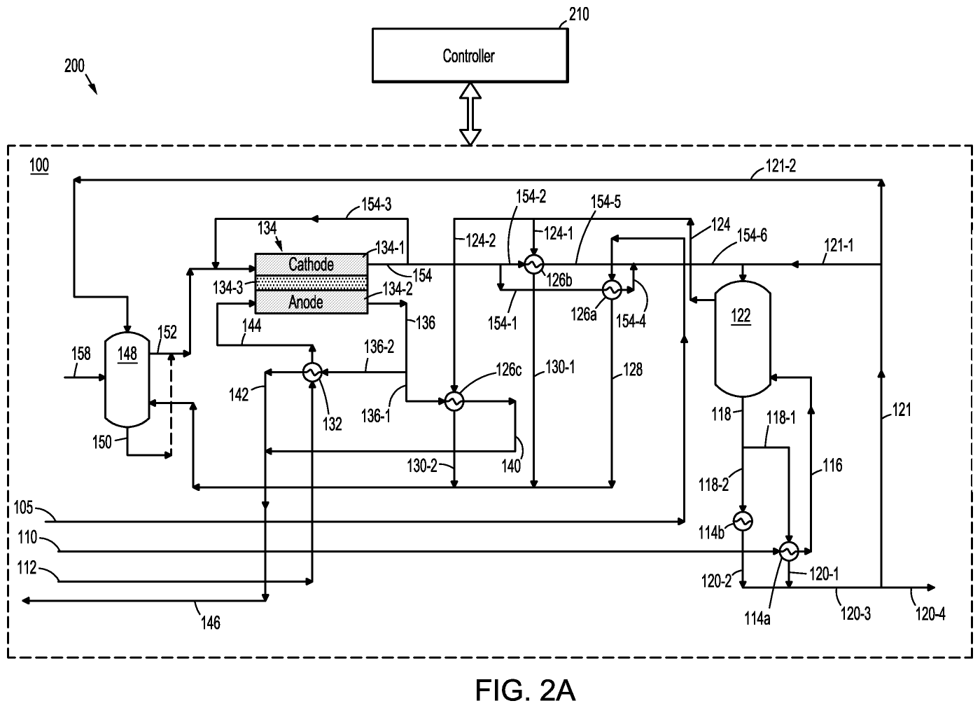

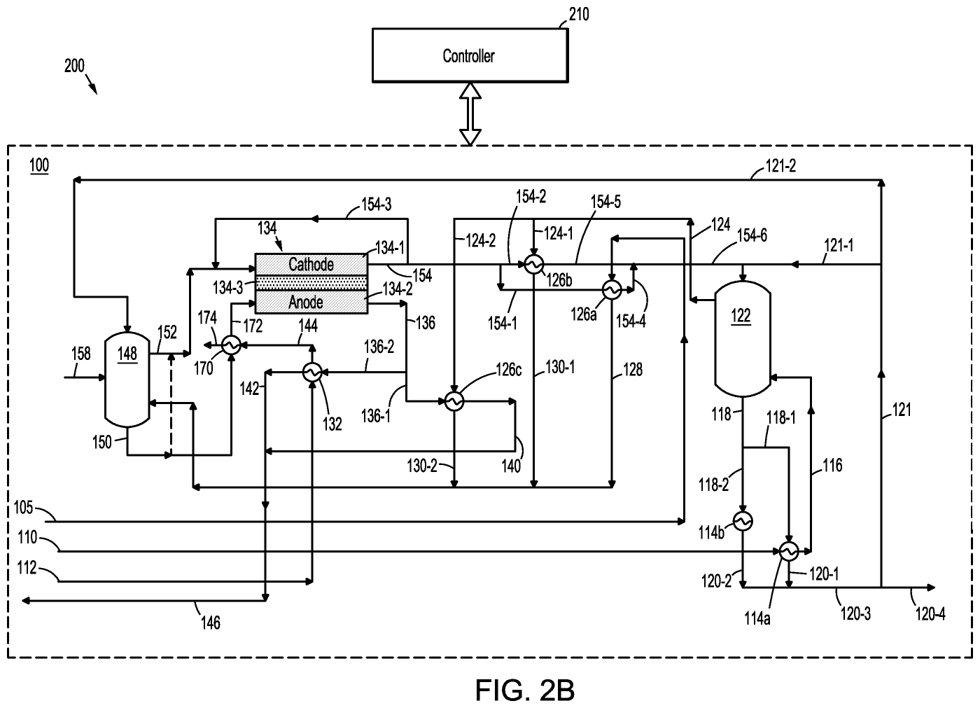

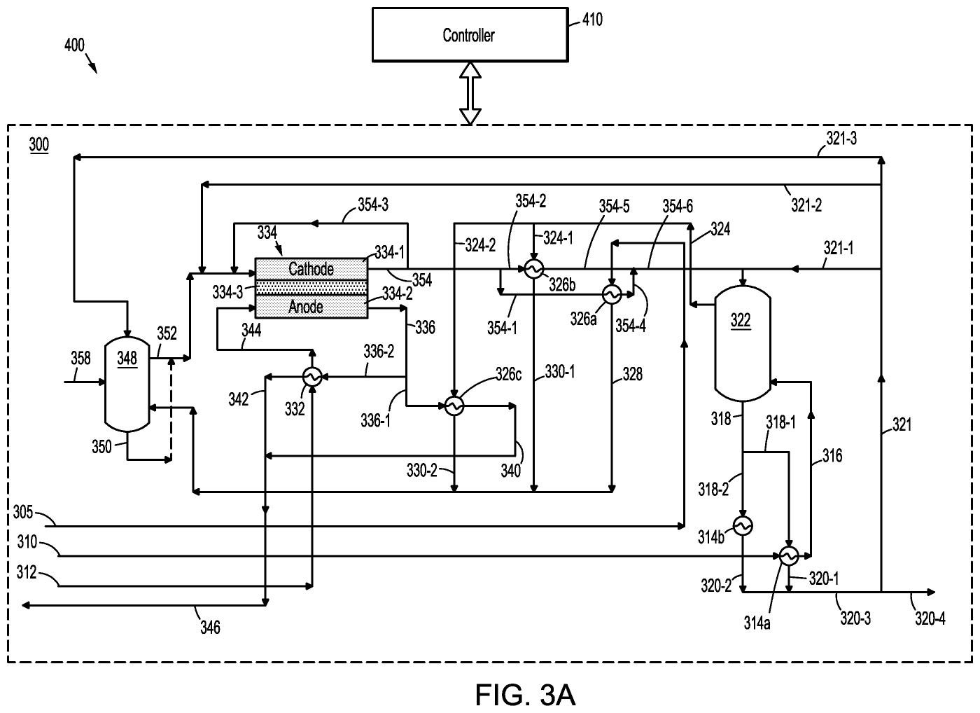

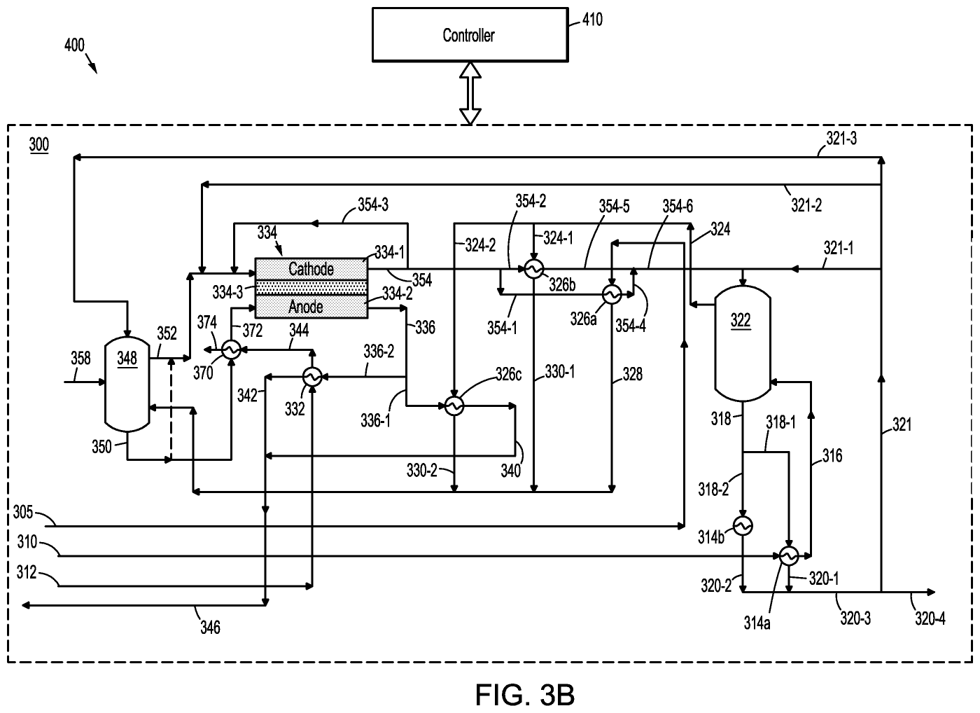

FIG. 1 illustrates a flow chart of method 10 for each of the starting feed streams including water feed 12, carbon dioxide feed 30 and anode purge stream 40 with heat integration design including a combustion unit for the electrochemical conversion of carbon dioxide and steam to produce syngas for production of methanol and dimethyl ether. In a non-limiting illustrative embodiment, the method utilizing the starting feed streams can be carried out in parallel. Method 10 for the electrochemical conversion of carbon dioxide and steam to syngas for the production of chemical products such as methanol and/or fuels according to the illustrative embodiments of the present disclosure will now be described with reference to FIGS. 2A-3B in combination with FIG. 1. For ease of understanding, specific examples mentioned in the following description are all illustrative and are not used to limit the scope of the present disclosure.

In an illustrative embodiment, one of the starting streams is water feed 12 at or around a temperature of about 20° C.

Step 14 of method 10 includes water feed 12 being heated by reactor synthesis effluent to generate a heated water stream having a temperature of 50° C. to about 150° C.

Step 16 of method 10 includes heated water stream being heated by reactor synthesis reaction heat to generate a steam feed stream having a temperature of from about 250° C. to about 350° C.

Step 18 of method 10 includes steam feed stream being split into two steam feeds and heating the first steam feed stream by an electrolyzer cathode effluent to generate a first heated steam effluent having a temperature of from about 550° C. to about 650° C., and heating the second steam feed stream by an electrolyzer anode effluent to generate a second heated steam effluent having a temperature of from about 550° C. to about 650° C.

Step 20 of method 10 includes first heated steam effluent and second heated steam effluent being heated by combustion of tail gas to generate a third heated steam effluent having a temperature of from about 700° C. to about 950° C.

Step 22 of method 10 includes sending the third heated steam effluent to the electrolyzer.

In an illustrative embodiment, one of the starting streams is carbon dioxide feed 30 at or around a temperature of about 20° C.

Step 32 of method 10 includes carbon dioxide feed 30 being heated by an electrolyzer cathode effluent to generate a heated carbon dioxide feed stream having a temperature of from about 550° C. to about 650° C.

Step 34 of method 10 includes heated carbon dioxide feed stream being heated by combustion of tail gas to generate another heated carbon dioxide feed stream having a temperature of from about 700° C. to about 950° C.

Step 36 of method 10 includes sending the other heated carbon dioxide feed stream to the electrolyzer.

In an illustrative embodiment, one of the starting streams is anode purge stream 40 at or around a temperature of about 20° C. As discussed below, representative examples of anode purge stream 40 include one or more of air, carbon dioxide or an inert gas such as N₂.

Step 42 of method 10 includes anode purge stream 40 being heated by an electrolyzer anode effluent to generate a heated anode purge stream.

Step 44 of method 10 includes heated anode purge stream being heated by an effluent of tail gas combustion.

Step 46 of method 10 includes sending the heated anode purge stream to the electrolyzer.

FIGS. 2A and 2B illustrate a method and system diagram with heat integration design for the electrochemical conversion of carbon dioxide and steam to produce syngas for production of methanol and dimethyl ether which can be further converted into other chemicals and/or fuels. Referring now to FIG. 2A, system 100 includes heat exchanger 114a for receiving water feed stream 110 at or around room temperature, i.e., about 20° C., and a first portion 118-1 of reactor synthesis effluent from reactor unit 122 as a heat transfer medium to generate a heated water stream 116 having a temperature of about 50° C. to about 150° C. and a cooled product effluent 120-1. In some embodiments, heat exchanger 114a may be a shell-and-tube, plate-fin, microchannel, spiral wound, or any other suitable heat exchanger. The reactor synthesis effluent from reactor unit 122 is a heated product effluent having a temperature of from about 250° C. to about 350° C. which is split into first portion 118-1 and second portion 118-2. Accordingly, first portion 118-1 of reactor synthesis effluent delivers the heat in heat exchanger 114a to water feed stream 110 to generate a heated water stream 116, and the first portion 118-1 of reactor synthesis effluent is likewise cooled against water feed stream 110 in heat exchanger 114a which cools first portion 118-1 of reactor synthesis effluent to generate a cooled product effluent 120-1, i.e., a temperature of less than 120° C. such as from about 80° C. to about 110° C.

System 100 further includes heat exchanger 114b for receiving second portion 118-2 of reactor synthesis effluent from reactor unit 122 and cooling second portion 118-2 of reactor synthesis effluent to generate a cooled reactor synthesis effluent 120-2 having a temperature of from about 80° C. to about 110° C. In some embodiments, heat exchanger 114b may be a shell-and-tube, plate-fin, microchannel, spiral wound, or any other suitable heat exchanger. In this particular embodiment, cooled product effluent 120-1 and cooled reactor synthesis effluent 120-2 are combined into reactor synthesis effluent 120-3 which contains such products as methanol, dimethyl ether, unreacted syngas, etc. The methanol or dimethyl ether products in reactor synthesis effluent 120-3 are separated out from unreacted syngas (also referred to as reactor tail gas) as reactor synthesis effluent 120-4 and sent for further downstream processing as known in the art. For example, reactor synthesis effluent 120-4 can be sent for further downstream processing for creating high value liquid fuels, such as gasoline, diesel, and jet. In some embodiments, heat exchanger 114b can also be used to heat the first reactor tail gas 121-1 before being recycled to the reactor 122.

Reactor tail gas stream 121 which contains mostly untreated syngas separated from reactor synthesis effluent 120-3 has a temperature of from about 80° C. to about 110° C. and is split into first reactor tail gas 121-1 and second reactor tail gas 121-2. First reactor tail gas 121-1 can be recycled back to reactor unit 122 for further processing as discussed above to enhance overall energy efficiency. Second reactor tail gas 121-2 is sent to the combustion unit 148 and combusted to generate thermal energy (i.e., heat) to transfer heat to the heated carbon dioxide effluent 128, first heated steam effluent 130-1 and second heated steam effluent 130-2 to generate a heated carbon dioxide and steam stream effluent 152 for sending to the cathode 134-1 of electrolyzer 134 as a continuous loop in the method and system of the illustrative embodiments as discussed below.

System 100 further includes reactor unit 122 for receiving heated water stream 116, first reactor tail gas 121-1, and cooled cathode effluent 154-6 as discussed below. In non-limiting illustrative embodiments, reactor unit 122 is a synthesis reactor for converting syngas to such desired products as methanol which can thereafter be converted to, for example, dimethyl ether, in the same or separate reactor (not shown) by conventional techniques, e.g., by methanol synthesis and in-situ dehydration, in which the in-situ methanol conversion can alleviate the thermodynamic limits of methanol synthesis, resulting in higher dimethyl ether yield. The produced methanol/dimethyl ether products can be purified and collected following conventional fractional distillation, while a portion of any unreacted syngas (also referred to as reactor tail gas) can be recycled (see first reactor tail gas 121-1) back to reactor unit 122 as discussed above.

The reaction in reactor unit 122 utilizing at least first reactor tail gas 121-1 and cooled cathode effluent 154-6 (i.e., a cooled syngas effluent) for making products such as, for example, methanol, is an exothermic reaction creating reaction heat to further heat and vaporize the incoming heated water stream 116 to generate a steam feed stream 124 having a temperature of from about 250° C. to about 350° C. In an illustrative embodiment, heated water stream 116 does not participate in the reaction. The reaction conditions and additional components in reactor unit 122 are within the purview of one skilled in the art. The products produced from the reaction process can then be discharged from reactor unit 122 as reactor synthesis effluent. Accordingly, for purposes of this illustrative embodiment, reactor synthesis effluent is one or more of methanol and/or dimethyl ether. However, this is merely illustrative and any other chemical product that can be made from the conversion of syngas is contemplated herein for use as reactor synthesis effluent.

System 100 further includes heat exchanger 126a for receiving carbon dioxide feed stream 105 and a first portion 154-1 of cathode effluent 154 comprised primarily of syngas, also referred to as heated cathode effluent 154, from the cathode 134-1 of electrolyzer 134 as a heat transfer medium to generate a heated carbon dioxide effluent 128 and a cooled cathode effluent 154-4. In some embodiments, heat exchanger 126a may be a shell-and-tube, plate-fin, microchannel, spiral wound, or any other suitable heat exchanger.

The received carbon dioxide feed stream 105 is a gas having a temperature of at or about 20° C. A carbon dioxide source in the present disclosure for carbon dioxide feed stream 105 can be obtained from several sources. For example, industrial manufacturing plants that produce ammonia for fertilizer produce large amounts of carbon dioxide. Ethanol plants that convert corn or wheat into ethanol produce large amounts of carbon dioxide. Power plants that generate electricity from various resources (e.g., natural gas, coal, other resources) produce large amounts of carbon dioxide. Chemical plants such as nylon production plants, ethylene production plants, other chemical plants produce large amounts of carbon dioxide. Some natural gas processing plants produce carbon dioxide as part of the process of purifying the natural gas to meet pipeline specifications. Capturing carbon dioxide for utilization as described herein often involves separating the carbon dioxide from a tail gas stream or another stream where the carbon dioxide is not the major component. Some carbon dioxide sources are already relatively pure and can be used with only minor treatment (which may include gas compression) in the methods described herein.

Referring back to cathode effluent 154, cathode effluent 154 exits the cathode 134-1 of electrolyzer 134 having a temperature of from about 700° C. to about 950° C. or from about 750° C. to about 850° C. and acts as a heat transfer medium in heat exchangers 126a and 126b. When exiting the cathode 134-1 of electrolyzer 134, cathode effluent 154 is split into first portion 154-1, second portion 154-2 and third portion 154-3. Accordingly, first portion 154-1 of cathode effluent 154 delivers the heat in heat exchanger 126a to carbon dioxide feed stream 105 which heats the carbon dioxide feed stream 105 to generate a heated carbon dioxide effluent 128 having a temperature of from about 550° C. to about 650° C., and the first portion 154-1 of cathode effluent 154 is likewise cooled against carbon dioxide feed stream 105 in heat exchanger 126a to generate a cooled cathode effluent 154-4, i.e., a temperature of less than 650° C. such as from about 250° C. to about 600° C.

System 100 further includes heat exchanger 126b for receiving a first portion 124-1 of steam feed stream 124 and second portion 154-2 of cathode effluent 154 from the cathode 134-1 of electrolyzer 134 as a heat transfer medium to generate a first heated steam effluent 130-1 and a cooled cathode effluent 154-5. In other words, second portion 154-2 of cathode effluent 154 delivers the heat in heat exchanger 126b to first portion 124-1 of steam feed stream 124 and generates a first heated steam effluent 130-1 having a temperature of from about 550° C. to about 650° C., and the second portion 154-2 of cathode effluent 154 is likewise cooled against first portion 124-1 of steam feed stream 124 in heat exchanger 126b to generate a cooled cathode effluent 154-5, i.e., a temperature of less than 650° C. such as from about 250° C. to about 600° C. Cooled cathode effluents 154-4 and 154-5 are then combined as cooled cathode effluent 154-6 and introduced to reactor unit 122 as discussed above. In some embodiments, heat exchanger 126b may be a shell-and-tube, plate-fin, microchannel, spiral wound, or any other suitable heat exchanger.

Third portion 154-3 of cathode effluent 154 is recycled back to cathode 134-1 where it is combined with incoming heated carbon dioxide and steam stream effluent 152 as discussed below.

System 100 further includes heat exchanger 126c for receiving a second portion 124-2 of steam feed stream 124 and a first portion 136-1 of anode effluent 136, i.e., an oxygen enriched stream or air effluent, from the anode 134-2 of electrolyzer 134 as a heat transfer medium to generate a second heated steam effluent 130-2 and a cooled anode effluent 140, i.e., a temperature of less than 650° C., such as from about 400° C. to about 620° C. As discussed below, anode effluent 136, also referred to as heated anode effluent 136, exits the anode 134-2 of electrolyzer 134 having a temperature of from about 700° C. to about 950° C. or from about 750° C. to about 850° C. and acts as a heat transfer medium in heat exchangers 126c and 132. Anode effluent 136 is split into first portion 136-1 and second portion 136-2. The first portion 136-1 of anode effluent 136 delivers the heat in heat exchanger 126c to heat second portion 124-2 of steam feed stream 124 and provide a second heated steam effluent 130-2 having a temperature of from about 550° C. to about 650° C., and the first portion 136-1 of anode effluent 136 is likewise cooled against second portion 124-2 of steam feed stream 124 in heat exchanger 126c to generate a cooled anode effluent 140. In some embodiments, heat exchanger 126c may be a shell-and-tube, plate-fin, microchannel, spiral wound, or any other suitable heat exchanger.

System 100 further includes heat exchanger 132 for receiving anode purge stream 112 and second portion 136-2 of anode effluent 136 from the anode 134-2 of electrolyzer 134 as a heat transfer medium to generate a heated anode purge stream 144 and a cooled anode effluent 142, i.e., a temperature of less than 650° C. such as from about 400° C. to about 620° C. In non-limiting illustrative embodiments, anode purge stream 112 is one of air, carbon dioxide or an inert gas such as N₂ having a temperature of at or about 20° C. In some embodiments, heat exchanger 132 may be a shell-and-tube, plate-fin, microchannel, spiral wound, or any other suitable heat exchanger. The received anode purge stream 112 will have a temperature of at or around about 20° C.

In some embodiments, the anode purge stream 112 such as air may be pressurized to produce a pressurized anode purge stream using fans, blowers, compressors or a combination thereof. The air compressor may be centrifugal, mixed-flow, axial-flow, reciprocating, rotary screw, rotary vane, scroll, diaphragm compressor, or a combination thereof. In some embodiments, the pressurized anode purge stream may have a pressure between about 1 bar to about 20 bar. In some embodiments, the pressurized anode purge stream may have a pressure of about 1 bar, about 1.2 bar, about 5 bar, about 10 bar, about 15 bar, or about 20 bar. In some embodiments, the pressurized anode purge stream may have a pressure of from about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar.

As discussed above, anode effluent 136 exits the anode 134-2 of electrolyzer 134 having a temperature of from about 700° C. to about 950° C. or from about 750° C. to about 850° C. and acts as a heat transfer medium in heat exchangers 126c and 132. Accordingly, second portion 136-2 of anode effluent 136 delivers the heat in heat exchanger 132 to heat anode purge stream 112 and provide a heated anode purge stream 144 having a temperature of from about 550° C. to about 650° C., and the second portion 136-2 of anode effluent 136 is likewise cooled against anode-forming stream 112 in heat exchanger 132 to generate a cooled anode effluent 142. In some embodiments, heated anode purge stream 144 can be further heated to about 750° C. to about 850° C. utilizing combusted stream 150 from the combustion unit 148 as discussed below.

Heated anode purge stream 144 is then sent to the anode 134-2 of electrolyzer 134 as discussed below. Cooled anode effluents 140 and 142 are then combined as cooled anode effluent 146 and sent for further processing. In an illustrative embodiment, when anode purge stream 112 is air, then cooled anode effluent 146 will be composed primarily of O₂ enrich with air, e.g., about 50% O₂ which can be utilized in a combustion process. As another example, when anode purge stream 112 is carbon dioxide, then cooled anode effluent 146 will be composed of CO₂ and O₂ which can be utilized in an oxy-combustion process.

System 100 further includes combustion unit 148 for receiving second reactor tail gas 121-2, heated carbon dioxide effluent 128, first heated steam effluent 130-1, second heated steam effluent 130-2 and oxidizing agent stream 158. Second reactor tail gas 121-2 is combusted with oxidizing agent stream 158 to transfer the heat generated in the combustion process to further heat the heated carbon dioxide effluent 128, first heated steam effluent 130-1 and second heated steam effluent 130-2 to generate a heated carbon dioxide and steam stream effluent 152 having a temperature of from about 700° C. to about 950° C. or from about 750° C. to about 850° C., and a combusted stream 150 (i.e., effluent of tail gas combustion) having a temperature of from about 700° C. to about 950° C. or from about 750° C. to about 850° C. In illustrative embodiments, the temperature in the combustion unit 148 can range from about 850° C. to about 1200° C. The heated carbon dioxide and steam stream effluent 152 is then sent to the cathode 134-1 of electrolyzer 134 as discussed below. The oxidizing agent stream 158 can be any suitable oxidizing source for combusting second reactor tail gas 121-2. In an illustrative embodiment, the oxidizing source is oxygen, air, or oxygen diluted with carbon dioxide (i.e., an oxygen enriched source).

As one skilled in the art will readily appreciate, the composition of combusted stream 150 depends on the particular oxidizing agent used in the combustion process. For example, in one case where the oxidizing source is air, then combusted stream 150 will contain carbon dioxide, nitrogen and unconverted oxygen. In this particular case, the combusted stream 150 as is cannot be sent to electrolyzer 134. Accordingly, in some embodiments, the combusted stream 150 will exit the combustion unit 148 for further processing. For example, in some embodiments, post treatment will be needed to separate nitrogen and unconverted oxygen to generate a purified carbon dioxide stream which can be combined with heated carbon dioxide and steam stream effluent 152 and sent to the cathode 134-1 of electrolyzer 134 as illustrated in FIG. 2A. In some embodiments, the oxidizing source is either oxygen or oxygen diluted with carbon dioxide such that the combusted stream 150 would contain predominantly carbon dioxide and minimal unconverted oxygen, and the combusted stream 150 can be combined with heated carbon dioxide and steam stream effluent 152 and sent to the cathode 134-1 of electrolyzer 134.

In an alternative embodiment, as depicted in FIG. 2B, system 100 further includes heat exchanger 170 for receiving heated anode purge stream 144 and combusted stream 150 as a heat transfer medium to generate a heated anode purge stream 172 having a temperature of from about 700° C. to about 950° C. or from about 750° C. to about 850° C. and a cooled combusted stream 174. In this particular embodiment, combusted stream 150 is generated when the oxidizing source is either oxygen or oxygen diluted with carbon dioxide such that the combusted stream 150 would contain predominantly carbon dioxide and minimal unconverted oxygen as discussed above. The heated anode purge stream 172 is then sent to the anode 134-2 of electrolyzer 134 to participate as a purge gas as discussed below. The cooled combusted stream 174 can be sent downstream for further processing as described above for combusted stream 150. In some embodiments, heat exchanger 170 may be a shell-and-tube, plate-fin, microchannel, spiral wound, or any other suitable heat exchanger.

The combustion unit 148 can be any combustion unit wherein a source of carbon can be combusted with oxygen. For example, the combustion unit 148 can be a fired heater or boiler used in power plants or a steam generator or a gas-fired turbine. Alternatively, the combustion unit 148 might be a coal fired combustion reactor. Those skilled in the art will appreciate that other combustion type reactors may also be utilized and are within the scope of the present disclosure.

System 100 further includes electrolyzer 134 for receiving heated carbon dioxide and steam stream effluent 152 having a temperature of from about 700° C. to about 950° C. or from about 750° C. to about 850° C. and third portion 154-3 of cathode effluent 154 having a temperature of from about 700° C. to about 950° C. or from about 750° C. to about 850° C. into cathode 134-1 and heated anode purge stream 144 (FIG. 2A) or heated anode purge stream 172 (FIG. 2B) into anode 134-2, where heated carbon dioxide and steam stream effluent 152 and third portion 154-3 of cathode effluent 154 participate in a reaction to generate a cathode (carbon monoxide (CO) and hydrogen (H₂)) effluent 154 from the cathode 134-1 and an anode effluent (oxygen enriched stream) 136 from the anode 134-2. Heated anode purge stream 144 (FIG. 2A) or heated anode purge stream 172 (FIG. 2B) serves as a purge gas to carry oxygen generated at the anode 134-2. In an illustrative embodiment, electrolyzer 134 can be any suitable high temperature electrolyzer comprising cathode 134-1, anode 134-2 and an electrolyte 134-3 inserted between the anode and the cathode. In a non-limiting illustrative embodiment, electrolyzer 134 is a high temperature solid oxide electrolyzer (also referred to as SOEC) comprising:
a first porous conductive electrode, or “cathode”, to be supplied with steam and carbon dioxide for the production of dihydrogen and carbon monoxide;
a second porous conductive electrode, or “anode”, via which the dioxygen (O₂) produced by the electrolysis of the water injected onto the cathode escapes; and
a solid oxide membrane (dense electrolyte) sandwiched between the cathode and the anode, the membrane being anionically conductive at high temperatures, usually temperatures above about 700° C. and up to about 950° C.

The electrolyzer 134 may receive input energy (i.e., electricity) from an intermittent source such as solar power (including photovoltaic and reflective), wind power, tidal power, wave power, batteries, and other intermittent energy sources known in the art and combinations thereof. Alternatively, or in addition, electrolyzer 134 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. The electrolyzer 134 may therefore be electricity connectable to an intermittent energy input, a non-intermittent source, or a combination thereof. In particular embodiments, electrolyzer 134 may receive input energy from the photovoltaic panel.

In some embodiments, electrolyzer 134 may be operational receiving electricity from a photovoltaic panel. At night, electrolyzer 134 may be operated in “hot stand-by” mode to conserve electricity, or electrolyzer 134 may be electricity connected to another power source to continue operating at night. In particular, electrolyzer 134 may be connected to a power grid such as a regional power grid, a municipal power grid, or a micro grid, and electrolyzer 134 may run when the price of electricity is low.

The system 100 may further comprise an energy storage mechanism or a plurality of energy storage mechanisms. The energy storage mechanism may comprise any mechanism or apparatus operable to store energy such as electricity, thermal energy, etc. For example, the energy storage mechanism may include batteries (e.g., lead-acid batteries, lithium-ion batteries, lithium iron batteries, etc.), ice, water, flywheels, compressed air, pumped hydroelectric, or other energy storage mechanisms known in the art and combinations thereof.

In illustrative embodiments, a solid oxide electrolyzer can include a stack of elementary solid-oxide (co-)electrolysis cells each comprising an anode, a cathode, and an electrolyte inserted between the anode and the cathode, the cells being electrically connected in series, the stack comprising two electrical terminals for the supply of current to the cells and defining flow chambers for, with respect to the first chambers, the flow of steam, hydrogen and carbon dioxide, and carbon monoxide over the cathodes and flow chambers for, with respect to the second chambers, the flow of air or nitrogen or oxygen or of a mixture of gases containing oxygen, and carbon dioxide over the anodes.

In illustrative embodiments, a solid oxide electrolyzer generally relies on an electron source (external source of electricity). The heat and electricity to operate the solid oxide electrolyzer may be produced from renewable sources, such as solar, wind, geothermal, or hydropower. Heat may be added to the solid oxide electrolyzer to maintain a desired operating temperature of the solid oxide electrolyzer including the electrochemical reduction. In an illustrative embodiment, heat may be added to the solid oxide electrolyzer by, for example, resistive heating (e.g., at the solid oxide electrolyzer electrodes), a steam jacket, solar heating systems, etc.

In some embodiments, the heated carbon dioxide and steam stream effluent 152 and third portion 154-3 of cathode effluent 154 participate in a reaction in a solid oxide electrolytic cell (SOEC) to generate a cathode effluent 154 and heated anode purge stream 144 (FIG. 2A) or heated anode purge stream 172 (FIG. 2B) participates as a purge gas. For example, the heated carbon dioxide and steam stream effluent 152 and third portion 154-3 of cathode effluent 154 may be reacted at a cathode 134-1 in electrolyzer 134 and the heated anode purge stream 144 (FIG. 2A) or heated anode purge stream 172 (FIG. 2B) participates as a purge gas at an anode 134-2 of electrolyzer 134 to generate cathode effluent 154 and anode effluent 136 including an oxygen enriched stream, where the cathode 134-1 and the anode 134-2 may be separated by an electrolyte 134-3. In some embodiments, the SOEC may operate at a temperature of about 700° C. to about 950° C. In some embodiments, the SOEC may operate at a temperature of about 700° C., about 820° C., about 730° C., about 740° C., about 750° C., about 760° C., about 770° C., about 780° C., about 790° C., about 800° C., about 810° C., about 820° C., about 830° C., about 840° C., about 860° C., about 880° C., about 900° C., about 910° C., about 920° C., about 930° C., about 940° C., or about 950° C., where any range from these limits are contemplated herein. In some embodiments, the cathode may operate at a temperature between about 750° C. to about 850° C., and the anode may operate at a temperature between about 750° C. to about 850° C.

In some embodiments, the SOEC may operate at a pressure between about 1 bar to about 20 bars. In some embodiments, the SOEC may operate at a pressure of about 1.02 bar, about 3 bar, about 5 bar, about 7 bar, about 9 bar, about 10 bar, about 15 bar or about 20 bar, where any range from these limits are contemplated herein. In some embodiments, the SOEC may operate at a pressure of about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar, where any range from these limits are contemplated herein.

The material of the solid oxide electrolyzer electrodes (i.e., cathode and anode) may be based on ceramic materials that exhibit stability through reduction-oxidation (redox) cycles, electrocatalytic activity, and mixed ionic and electronic conductivity in reducing atmospheres. The material of the solid oxide electrodes may be metal or metal oxide-based material (e.g., Ni-based electrodes). In some embodiments, the cathode and anode may be constructed of any suitable material including, for example, (La,Sr) (Fe,Co)O₃(LSCF), (Sm,Sr)CoO₃, and Sr-doped LaMnO₃ for the anode electrode (anode) and Ni—YSZ, Ni—ScSZ, La₂NiO₄, and Ni—ZrO₂ for the cathode electrode. Electrode support materials and functional layers include nickel cermets, and other electronic conductors such as (Sr_{0.8}La_{0.2})TiO₃(SLT). The electrolyte may be comprised of any suitable material such as, for example, yttria-stabilized zirconia (YSZ), (La_{0.6}Sr_{0.4})(Ga_{0.8}Mg_{0.2})O₃(LSGM), Sc-stabilized zirconia (SSZ), and doped ceria. A SOEC cell architecture includes both electrode- and electrolyte-supported cell constructions and ceramic or metallic interconnects.

The electrolyzer 134 can operate in a co-electrolysis mode in which heated carbon dioxide and steam stream effluent 152 and third portion 154-3 of cathode effluent 154 are fed to the cathode 134-1 of electrolyzer 134. The mixture output from the cathode 134-1 is then composed of syngas including hydrogen (H₂) and carbon monoxide (CO) and small portion of unreacted carbon dioxide and steam as cathode effluent 154. The co-electrolysis mode further includes heated anode purge stream 144 being fed to the anode 134-2 of electrolyzer 134. The output from the anode is then composed of an oxygen enriched stream including oxygen (O₂) as anode effluent 136. In some embodiments, the cooled cathode effluent 154-6 can be compressed, cooled, and dehydrated before feeding to reactor unit 122.

In one or more illustrative embodiments, as depicted in FIGS. 2A and 2B, a system processing environment 200 comprises each of the components of system 100 described herein, as well as a controller 210 operatively coupled to system 100. Controller 210 is configured to control operations of one or more of the components of system 100 discussed above. In one illustrative embodiment, controller 210 is configured to actuate one or more of the functionalities of system 100 described herein. For example, controller 210 can comprise one or more processing devices configured to load software instructions from one or more memory devices and execute the software instructions to generate data and/or control signals that can be applied to one or more components of system 100 so as to actuate the functionalities described herein. Actuation of the components by the data and/or control signals may be affected electrically, electromechanically, electrochemically, and/or the like, depending on the nature of the specific component of system 100 being actuated.

Thus, in some embodiments, controller 210 comprises a combination of hardware and software components. For example, the one or more processing devices of controller 210 may comprise one or more microprocessors, one or more microcontrollers, one or more application-specific devices, or other types of processing circuitry, as well as portions or combinations thereof. Further, the one or more memory devices of controller 210 may comprise random access memory (RAM), read-only memory (ROM), or other types of memory, in any combination. It is to be appreciated that the specific architecture of controller 210 is configurable based on the components of system 100 and the functionalities they are intended to perform.

For example, controller 210 can be operatively connected to a processing device in a processing platform which comprises a processor coupled to a memory. The processor may comprise a microprocessor, a microcontroller, an application-specific integrated circuit (ASIC), a field-programmable gate array (FPGA) or other type of processing circuitry, as well as portions or combinations of such circuitry elements. The memory may comprise random access memory (RAM), read-only memory (ROM) or other types of memory, in any combination.

A system and process for the electrochemical conversion of water and carbon dioxide to produce a mixture of H₂ and CO (i.e., syngas), which is then fed to a Fischer-Tropsch (FT) reactor unit for fuel synthesis utilizing heat sources will now be described with reference to FIGS. 3A and 3B.

Referring now to FIGS. 3A and 3B, system 300 includes heat exchanger 314a for receiving water feed stream 310 at or around room temperature, i.e., about 20° C., and a first portion 318-1 of Fischer-Tropsch reactor synthesis effluent 318 from Fischer-Tropsch reactor unit 322 as a heat transfer medium to generate a heated water stream 316 having a temperature of about 50° C. to about 150° C. and a cooled Fischer-Tropsch reactor synthesis effluent 320-1. In some embodiments, heat exchanger 314a may be a shell-and-tube, plate-fin, microchannel, spiral wound, or any other suitable heat exchanger. The Fischer-Tropsch reactor synthesis effluent 318 from reactor unit 322 is a Fischer-Tropsch reactor synthesis effluent 318 having a temperature of from about 250° C. to about 350° C. which is split into first portion 318-1 and second portion 318-2. Accordingly, first portion 318-1 of Fischer-Tropsch reactor synthesis effluent 318 delivers the heat in heat exchanger 314a to water feed stream 310 to generate heated water stream 316, and the first portion 318-1 of Fischer-Tropsch reactor synthesis effluent 318 is likewise cooled against water feed stream 310 in heat exchanger 314a which cools first portion 318-1 of Fischer-Tropsch reactor synthesis effluent 318 to generate cooled Fischer-Tropsch reactor synthesis effluent 320-1, i.e., a temperature of less than 300° C. such as from about 200° C. to about 280° C.

System 300 further includes heat exchanger 314b for receiving second portion 318-2 of Fischer-Tropsch reactor synthesis effluent 318 from Fischer-Tropsch reactor unit 322 and cooling second portion 318-2 of Fischer-Tropsch reactor synthesis effluent 318 to generate a cooled Fischer-Tropsch reactor synthesis effluent 320-2 having a temperature of from about 200° C. to about 280° C. In some embodiments, heat exchanger 314b can be any heat exchanger as discussed above for heat exchanger 114b. In this particular embodiment, cooled Fischer-Tropsch reactor synthesis effluent 320-1 and cooled Fischer-Tropsch reactor synthesis effluent 320-2 are combined into Fischer-Tropsch reactor synthesis effluent 320-3 which contains Fischer-Tropsch products. For example, the Fischer-Tropsch process involves a series of chemical reactions that produce a variety of hydrocarbons, ideally having the formula (CₙH₂ₙ₊₂). In an illustrative embodiment, the more useful reactions produce alkanes such as follows: (2n+1) H₂+n CO→CₙH₂ₙ₊₂+nH₂O where n is from 1 to 70. In some embodiments, heat exchanger 314b can also be used to heat the first reactor tail gas 321-1 before being recycled to the reactor 322.

In an illustrative embodiment, the Fischer-Tropsch reactor synthesis effluent 318 resulting in the Fischer-Tropsch reactor synthesis effluent 320-3 contains hydrocarbon product such as a C₁ to C₇₀ hydrocarbon product as the key product together with unreacted syngas and byproducts such as carbon dioxide. In some embodiments, the system may include a hydrocracker unit and/or fractionation unit (not shown) to upgrade the Fischer-Tropsch liquids. For example, the hydrocracker unit employs a high temperature, high pressure catalytic process that can upgrade heavy Fischer-Tropsch liquid (HFTL) and medium Fischer-Tropsch liquid (MFTL) hydrocarbon streams into a transportation fuel or a blending component meeting chemical and physical properties.

Accordingly, the Fischer-Tropsch reactor synthesis effluent 320-3 containing the resulting hydrocarbon products are separated out from the unreacted syngas and byproducts as Fischer-Tropsch reactor synthesis effluent 320-4 and sent for further downstream processing as known in the art. For example, Fischer-Tropsch reactor synthesis effluent 320-4 can be sent for further downstream processing for creating high value liquid fuels, such as gasoline, diesel, and jet. The reactor tail gas 321 composed of unreacted syngas and gaseous byproducts separated from Fischer-Tropsch reactor synthesis effluent 320-3 is split into first reactor tail gas 321-1, carbon dioxide enriched tail gas stream 321-2 and second reactor tail gas 321-3. First reactor tail gas 321-1 is recycled back to the Fischer-Tropsch reactor unit 322 for further Fischer-Tropsch synthesis to increase the overall carbon efficiency. Carbon dioxide enriched tail gas stream 321-2 composed primarily of carbon dioxide and methane is recycled back to the cathode 334-1 of electrolyzer 334 for further syngas generation via electrolysis. Second reactor tail gas stream 321-3 is sent to the combustion unit 348 and combusted as discussed above to generate thermal energy and transfer heat to the heated carbon dioxide effluent 328, first heated steam effluent 330-1 and second heated steam effluent 330-2 to generate a heated carbon dioxide and steam stream effluent 352 for sending to the cathode 334-1 of electrolyzer 334 as a continuous loop in the method and system of the illustrative embodiments as discussed below.

System 300 further includes Fischer-Tropsch reactor unit 322 for receiving heated water stream 316, first reactor tail gas 321-1, and cooled cathode effluent 354-6 as discussed below. In non-limiting illustrative embodiments, Fischer-Tropsch reactor unit 322 is a Fischer-Tropsch reactor for receiving first reactor tail gas 321-1, and cooled cathode effluent 354-6 which participate in a reaction to convert syngas to a Fischer-Tropsch product by conventional techniques. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. In illustrative embodiments, the heated water stream 316 does not participate in the reaction of first reactor tail gas 321-1 and cooled cathode effluent 354-6.

Numerous types of reactor systems have been developed for carrying out the Fischer-Tropsch reaction. For example, Fischer-Tropsch reactor systems include fixed bed reactors, especially multitubular fixed bed reactors, fluidized bed reactors, such as entrained fluidized bed reactors and slurry bed reactors such as three-phase slurry bubble columns and ebullated bed reactors. The present invention is applicable to all types of reactor systems. The reactors each have an inlet for receiving synthesis gas and an outlet for discharging an effluent stream.

The Fischer-Tropsch synthesis utilizing first reactor tail gas 321-1 and cooled cathode effluent 354-6 is an exothermic reaction creating reaction heat to further heat and vaporize the incoming heated water stream 316 to generate a steam feed stream 324 having a temperature of from about 250° C. to about 350° C. The hydrocarbon products produced from the Fischer-Tropsch process are then discharged from reactor unit 322 as Fischer-Tropsch reactor synthesis effluent 318.

System 300 further includes heat exchanger 326a for receiving carbon dioxide feed stream 305 and a first portion 354-1 of cathode effluent 354, also referred to as heated cathode effluent 354, from the cathode 334-1 of electrolyzer 334 as a heat transfer medium to generate a heated carbon dioxide effluent 328 and a cooled cathode effluent 354-4. In some embodiments, heat exchanger 326a can be any heat exchanger as discussed above for heat exchanger 126a. The received carbon dioxide feed stream 305 is a gas having a temperature of at or about 20° C. A carbon dioxide source in the present disclosure for carbon dioxide feed stream 305 can be obtained from any of the sources discussed above for carbon dioxide feed stream 105.

Referring back to cathode effluent 354, cathode effluent 354 exits the cathode 334-1 of electrolyzer 334 having a temperature of from about 700° C. to about 950° C. or from about 750° C. to about 850° C. and acts as a heat transfer medium in heat exchangers 326a and 326b. Cathode effluent 354 from the cathode 334-1 of electrolyzer 334 is split into first portion 354-1, second portion 354-2 and third portion 354-3. Accordingly, first portion 354-1 of cathode effluent 354 delivers the heat in heat exchanger 326a to carbon dioxide feed stream 305 which heats the carbon dioxide feed stream 305 to generate a heated carbon dioxide effluent 328 having a temperature of from about 550° C. to about 650° C., and the first portion 354-1 of cathode effluent 354 is likewise cooled against carbon dioxide feed stream 305 in heat exchanger 326a to generate a cooled cathode effluent 354-4, i.e., a temperature of less than 650° C. such as from about 250° C. to about 600° C. In some embodiments, heat exchanger 326a can be any of those discussed above for heat exchanger 126a.

System 300 further includes heat exchanger 326b for receiving a first portion 324-1 of steam feed stream 324 and second portion 354-2 of cathode effluent 354 from the cathode 334-1 of electrolyzer 334 as a heat transfer medium to generate a first heated steam effluent 330-1 and a cooled cathode effluent 354-5. In other words, second portion 354-2 of cathode effluent 354 delivers the heat in heat exchanger 326b to heat first portion 324-1 of steam feed stream 324 and generate a first heated steam effluent 330-1 having a temperature of from about 550° C. to about 650° C., and the second portion 354-2 of cathode effluent 354 is likewise cooled against first portion 324-1 of steam feed stream 324 in heat exchanger 326b to generate a cooled cathode effluent 354-5, i.e., a temperature of less than 650° C. such as from about 250° C. to about 600° C. Cooled cathode effluents 354-4 and 354-5 are then combined as cooled cathode effluent 354-6 and introduced to Fischer-Tropsch reactor unit 322 for further processing as discussed above. In some embodiments, heat exchanger 326b can be any of those discussed above for heat exchanger 126b.

Third portion 354-3 of cathode effluent 354 can be recycled back to cathode 334-1 where it is combined with heated carbon dioxide and steam stream effluent 352 as discussed below.

System 300 further includes heat exchanger 326c for receiving a second portion 324-2 of steam feed stream 324 and a first portion 336-1 of anode effluent 336, i.e., an oxygen enriched stream from the anode 334-2 of electrolyzer 334 as a heat transfer medium to generate a second heated steam effluent 330-2 and a cooled anode effluent 340. In other words, anode effluent 336, also known as heated anode effluent 336, exits the anode 334-2 of electrolyzer 334 having a temperature of from about 700° C. to about 950° C. or from about 750° C. to about 850° C. and acts as a heat transfer medium in heat exchangers 326c and 332.

Anode effluent 336 is split into first portion 336-1 and second portion 336-2. In some embodiments, first portion 336-1 of anode effluent 336 delivers the heat in heat exchanger 326c to heat second portion 324-2 of steam feed stream 324 and provide a second heated steam effluent 330-2 having a temperature of from about 550° C. to about 650° C., and the first portion 336-1 of anode effluent 336 is likewise cooled against second portion 324-2 of steam feed stream 324 in heat exchanger 326c to generate a cooled anode effluent 340, i.e., a temperature of less than 650° C. such as from about 400° C. to about 620° C. In some embodiments, heat exchanger 326c can be any heat exchanger as discussed above for heat exchanger 126c.

System 300 further includes heat exchanger 332 for receiving anode purge stream 312 and second portion 336-2 of anode effluent 336 from the anode 334-2 of electrolyzer 334 as a heat transfer medium to generate a heated anode purge stream 344 and a cooled anode effluent 342. The received anode purge stream 312 will have a temperature of at or around about 20° C. In some embodiments, the anode purge stream 312 may be pressurized as discussed above for anode purge stream 112. In some embodiments, heat exchanger 332 can be any of those discussed above for heat exchanger 132. The received anode purge stream 312 will have a temperature of at or around about 20° C. Anode purge stream 312 can be of similar streams as anode purge stream 112 discussed above.

As discussed above, anode effluent 336 exits the anode 334-2 of electrolyzer 334 having a temperature of from about 700° C. to about 950° C. or from about 750° C. to about 850° C. and acts as a heat transfer medium in heat exchangers 326c and 332. In some embodiments, second portion 336-2 of anode effluent 336 delivers the heat in heat exchanger 332 to heat anode purge stream 312 and provide a heated anode purge stream 344 having a temperature of from about 550° C. to about 650° C., and the second portion 336-2 of anode effluent 336 is likewise cooled against anode purge stream 312 in heat exchanger 332 to generate a cooled anode effluent 342, i.e., a temperature of less than 650° C. such as from about 400° C. to about 620° C. In an embodiment, heated anode purge stream 344 is then sent to the anode 334-2 of electrolyzer 334 as discussed below. In an embodiment, heated anode purge stream 344 can be further heated utilizing combusted stream 350 as discussed below.

Cooled anode effluents 340 and 342 are then combined as cooled anode effluent 346 and sent for further processing. In an illustrative embodiment, when anode purge stream 312 is air, then cooled anode effluent 346 will be composed primarily of O₂ enrich with air, e.g., about 50% O₂ which can be utilized in a combustion process. As another example, when anode purge stream 312 is carbon dioxide, then cooled anode effluent 346 will be composed of CO₂ and O₂ which can be utilized in an oxy-combustion process.

System 300 further includes combustion unit 348 for receiving second reactor tail gas 321-3, heated carbon dioxide effluent 328, first heated steam effluent 330-1, second heated steam effluent 330-2 and oxidizing agent stream 358. Second reactor tail gas 321-3 is combusted with oxidizing agent stream 358 to generate heat to further heat the heated carbon dioxide effluent 328, first heated steam effluent 330-1 and second heated steam effluent 330-2 thereby generating a heated carbon dioxide and steam stream effluent 352 having a temperature of about 700° C. to about 950° C. or from about 750° C. to about 850° C., and a combusted stream 350 (i.e., effluent of tail gas combustion) having a temperature of from about 700° C. to about 950° C. or from about 750° C. to about 850° C. In illustrative embodiments, the temperature in the combustion unit 148 can range from about 850° C. to about 1200° C. The heated carbon dioxide and steam stream effluent 352 is then sent to the cathode 334-1 of electrolyzer 334 as discussed above. The oxidizing agent stream 358 can be any suitable oxidizing source as discussed above for oxidizing agent stream 158.

As one skilled in the art will readily appreciate, the composition of combusted stream 350 depends on the particular oxidizing agent used in the combustion process. For example, in one case where the oxidizing source is air, then combusted stream 350 will contain carbon dioxide, nitrogen and unconverted oxygen. In this particular case, the combusted stream 350 as is cannot be sent to electrolyzer 334. Accordingly, in some embodiments, the combusted stream 350 will exit the combustion unit 348 for further processing. For example, in some embodiments, post treatment will be needed to separate nitrogen and unconverted oxygen to generate a purified carbon dioxide stream which can be combined with heated carbon dioxide and steam stream effluent 352 and sent to the cathode 334-1 of electrolyzer 334 as illustrated in FIG. 3A. In some embodiments, the oxidizing source is either oxygen or oxygen diluted with carbon dioxide such that the combusted stream 350 would contain predominantly carbon dioxide and minimal unconverted oxygen, and the combusted stream 350 can be combined with heated carbon dioxide and steam stream effluent 352 and sent to the cathode 334-1 of electrolyzer 334.

In an alternative embodiment, as depicted in FIG. 3B, system 300 further includes heat exchanger 370 for receiving heated anode purge stream 344 and combusted stream 350 as a heat transfer medium to generate a heated anode purge stream 372 having a temperature of from about 700° C. to about 950° C. or from about 750° C. to about 850° C. and a cooled combusted stream 374. In this particular embodiment, combusted stream 350 is generated when the oxidizing source is either oxygen or oxygen diluted with carbon dioxide such that the combusted stream 350 would contain predominantly carbon dioxide and minimal unconverted oxygen as discussed above. The heated anode purge stream 372 is then sent to the anode 334-2 of electrolyzer 334 to participate as a purge gas as discussed below. The cooled combusted stream 374 can be sent downstream for further processing as described above for combusted stream 350. In some embodiments, heat exchanger 370 can be any of those discussed above for heat exchanger 170.

System 300 further includes electrolyzer 334 for receiving heated carbon dioxide and steam stream effluent 352 having a temperature of from about 700° C. to about 950° C. or from about 750° C. to about 850° C. and third portion 354-3 of cathode effluent 354 having a temperature of from about 700° C. to about 950° C. or from about 750° C. to about 850° C. into cathode 334-1 to participate in a reaction to generate corresponding cathode effluent 354 including syngas composed of carbon monoxide (CO) and hydrogen (H₂) and heated anode purge stream 344 (FIG. 3A) or heated anode purge stream 372 (FIG. 3B) into anode 334-2, where heated carbon dioxide and steam stream effluent 352 and third portion 354-3 of cathode effluent 354 participate in a reaction to generate a cathode (carbon monoxide (CO) and hydrogen (H₂)) effluent 354 from the cathode 334-1 and an anode effluent (oxygen enriched stream) 336 from the anode 334-2. Heated anode purge stream 344 (FIG. 3A) or heated anode purge stream 372 (FIG. 3B) serves as a purge gas to carry oxygen generated at the anode 334-2. The electrolyzer 334 can be any suitable high temperature electrolyzer as discussed above for electrolyzer 134.

In some embodiments, the heated carbon dioxide and steam stream effluent 352 and third portion 354-3 of cathode effluent 354 participate in a reaction in a solid oxide electrolytic cell (SOEC) (electrolyzer 334) to generate a cathode effluent 354 and heated anode purge stream 344 (FIG. 3A) or heated anode purge stream 372 (FIG. 3B) participates as a purge gas. For example, the heated carbon dioxide and steam stream effluent 352 may be reacted at a cathode 334-1 in electrolyzer 334 and the heated anode purge stream 344 (FIG. 3A) or heated anode purge stream 372 (FIG. 3B) participates as purge gas purge the at an anode 334-2 of electrolyzer 334 to generate cathode effluent 354 and anode effluent 336, where the cathode 334-1 and the anode 334-2 may be separated by an electrolyte 334-3. In an illustrative embodiment, electrolyzer 334 can be any suitable high temperature electrolyzer comprising cathode 334-1, anode 334-2 and an electrolyte 334-3 inserted between the anode and the cathode as discussed above for electrolyzer 134. In some embodiments, the SOEC may operate at a temperature and pressure discussed above for electrolyzer 134. For example, in illustrative embodiments, the cathode may operate at a temperature between about 750° C. to about 850° C., and the anode may operate at a temperature between about 750° C. to about 850° C. The material of the solid oxide electrolyzer electrodes can be any of those discussed above.

The electrolyzer 334 can operate in a co-electrolysis mode in which heated carbon dioxide and steam stream effluent 352 composed of steam (H₂O) and carbon dioxide (CO₂) and third portion 354-3 of cathode effluent 354 are fed to the cathode 334-1 of electrolyzer 334. The mixture output from the cathode is then composed of syngas composed of mainly hydrogen (H₂) and carbon monoxide (CO) as cathode effluent 354 and small portion of unconverted CO₂ and steam. The co-electrolysis mode further includes heated anode purge stream 344 fed to the anode 334-2 of electrolyzer 334. The output from the anode is then composed of an oxygen enriched stream including oxygen (O₂) as anode effluent 336. In some embodiments, the cooled cathode effluent 354-6 can be compressed, cooled, and dehydrated before feeding to Fischer Tropsch reactor unit 322.

In one or more illustrative embodiments, as depicted in FIGS. 3A and 3B, a system processing environment 400 comprises each of the components of system 300 described herein, as well as a controller 410 operatively coupled to system processing environment 400. Controller 410 is configured to control operations of one or more of the components of system 300 discussed above. In one illustrative embodiment, controller 410 is configured to actuate one or more of the functionalities of system 300 described herein. For example, controller 410 can be similar to controller 210 described above and function in a similar manner.

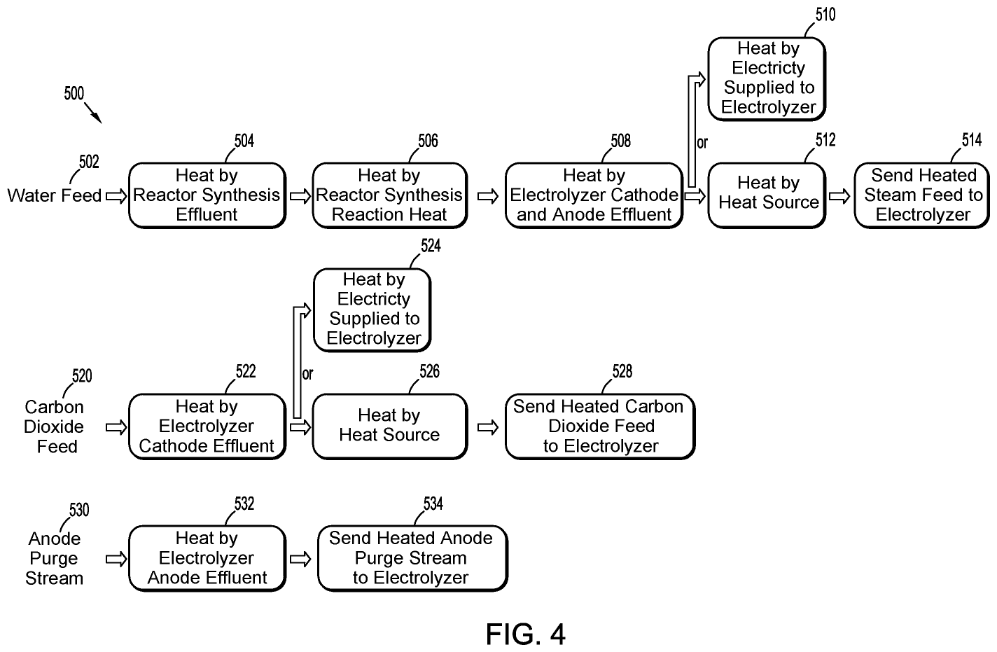

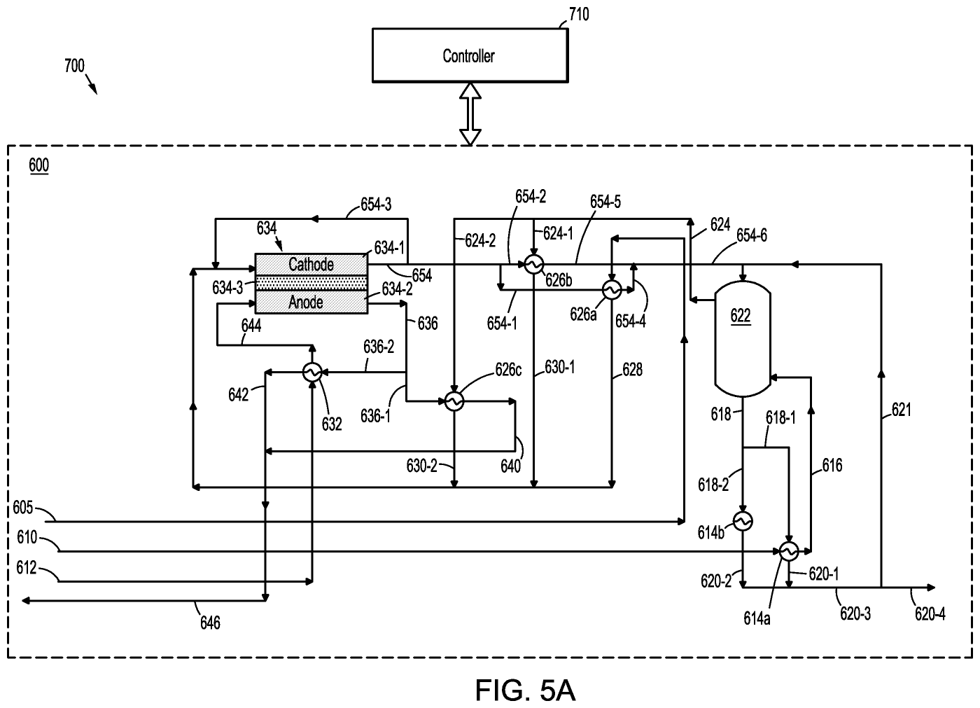

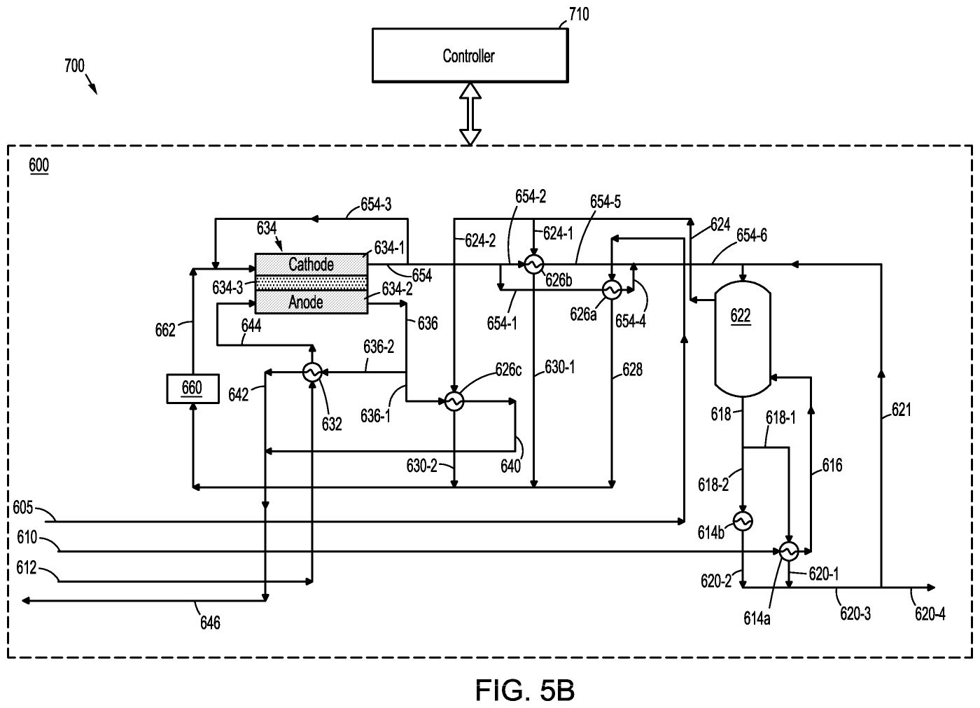

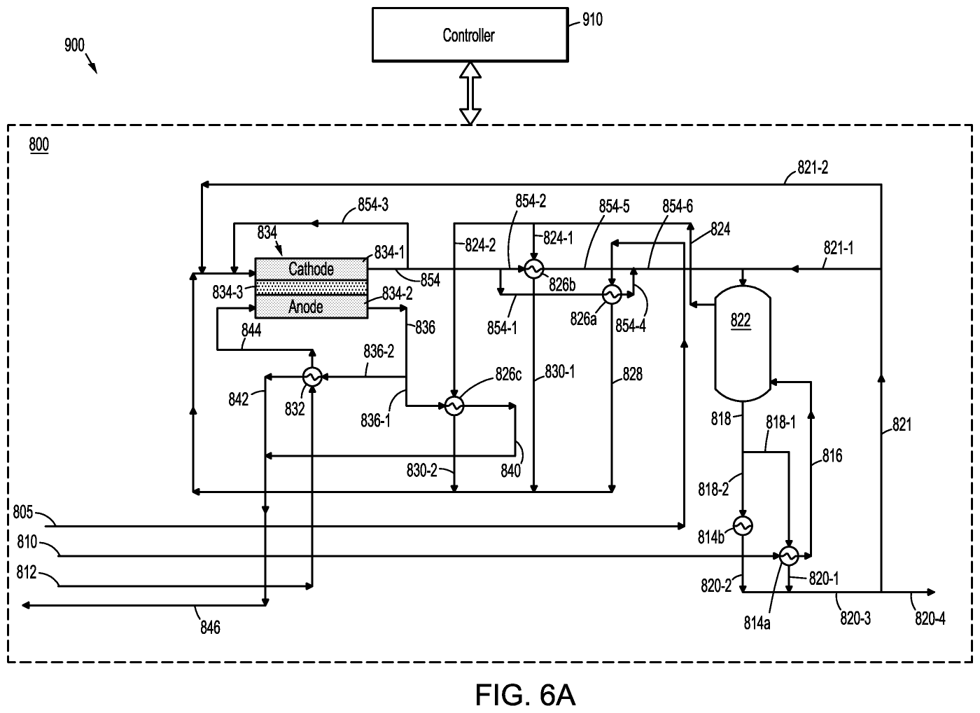

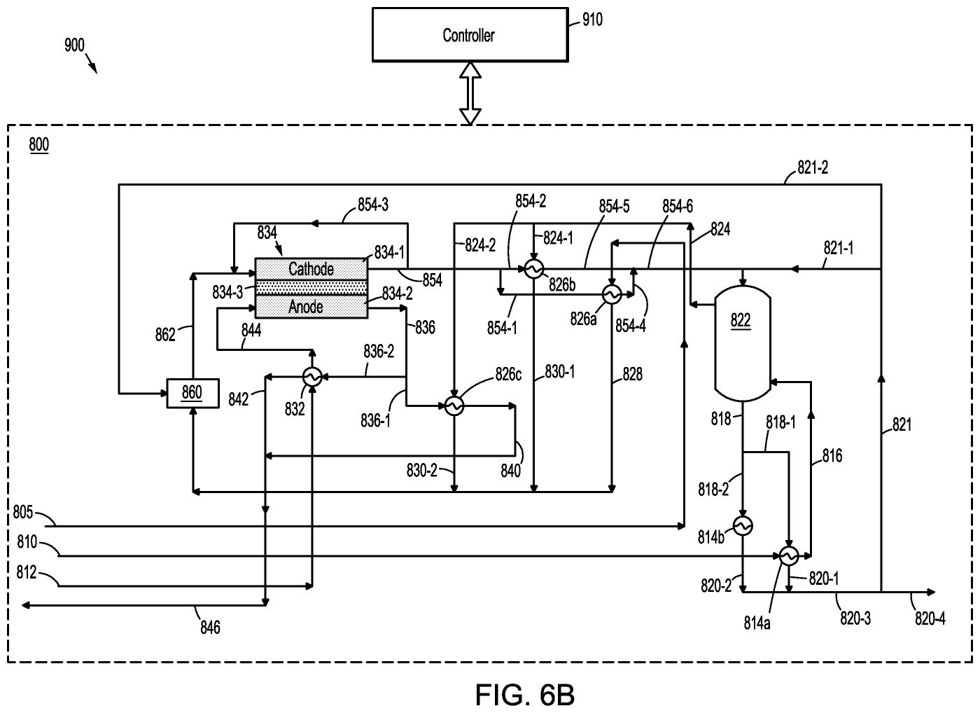

FIG. 4 illustrates a flow chart of method 500 for each of the starting feed streams including water feed 502, carbon dioxide feed 520 and anode purge stream 530 with heat integration design not including a combustion unit for the electrochemical conversion of carbon dioxide and steam to produce syngas for production of methanol and dimethyl ether. In a non-limiting illustrative embodiment, the method utilizing the starting feed streams can be carried out in parallel. Method 500 for the electrochemical conversion of carbon dioxide and steam to syngas for the production of chemical products such as methanol and/or fuels according to the illustrative embodiments of the present disclosure will now be described with reference to FIGS. 5A-6B in combination with FIG. 4. For case of understanding, specific examples mentioned in the following description are all illustrative and are not used to limit the scope of the present disclosure.

In an illustrative embodiment, one of the starting streams is water feed 502 at or around a temperature of about 20° C.

Step 504 of method 500 includes water feed 502 being heated by reactor synthesis effluent to generate a heated water stream having a temperature of 50° C. to about 150° C.

Step 506 of method 500 includes heated water stream being heated by reactor synthesis reaction heat to generate a steam feed stream having a temperature of from about 250° C. to about 350° C.

Step 508 of method 500 includes steam feed stream being split into two steam feeds and heating the first steam feed by an electrolyzer cathode effluent to generate a first heated steam effluent having a temperature of from about 550° C. to about 650° C., and heating the second steam feed stream by an electrolyzer anode effluent to generate a second heated steam effluent having a temperature of from about 550° C. to about 650° C.

The first heated steam effluent and second heated steam effluent are then subjected to either one of steps 510 or 512. Step 510 of method 500 includes first heated steam effluent and second heated steam effluent being heated by additional electricity supplied to the electrolyzer to generate a third heated steam effluent having a temperature of from about 700° C. to about 950° C.

Alternative step 512 of method 500 includes first heated steam effluent and second heated steam effluent being heated by a heat source to generate a third heated steam effluent having a temperature of from about 700° C. to about 950° C.

Step 514 of method 500 includes sending the third heated steam feed stream from step 512 to the electrolyzer.

In an illustrative embodiment, one of the starting streams is carbon dioxide feed 520 at or around a temperature of about 20° C.

Step 522 of method 500 includes carbon dioxide feed 520 being heated by an electrolyzer cathode effluent to generate a heated carbon dioxide feed stream having a temperature of from about 550° C. to about 650° C.

The heated carbon dioxide feed stream is then subjected to either one of steps 524 or 526. Step 524 of method 500 includes heated carbon dioxide feed stream being heated by additional electricity supplied to the electrolyzer to generate a second heated carbon dioxide feed stream having a temperature of from about 700° C. to about 950° C.

Alternative step 526 of method 500 includes heated carbon dioxide feed stream being heated by a heat source to generate a second heated carbon dioxide feed stream having a temperature of from about 700° C. to about 950° C.

Step 528 of method 500 includes sending the second heated carbon dioxide feed stream from step 526 to the electrolyzer.

In an illustrative embodiment, one of the starting streams is anode purge stream 530 at or around a temperature of about 20° C. As discussed below, representative examples of anode purge stream 530 include one or more of air, carbon dioxide or an inert gas such as N₂.

Step 532 of method 500 includes anode purge stream being heated by an electrolyzer anode effluent to generate a heated anode purge stream.

Step 534 of method 500 includes sending the heated anode purge stream to the electrolyzer.

FIGS. 5A-6B illustrate an alternative non-limiting illustrative embodiment with heat integration design without a combustion unit for electrochemical conversion of carbon dioxide into syngas. Referring now to FIG. 5A, system 600 includes heat exchanger 614a for receiving water feed stream 610 at or around room temperature, i.e., about 20° C., and a first portion 618-1 of reactor synthesis effluent 618 from reactor unit 622 as a heat transfer medium to generate a heated water stream 616 having a temperature of about 50° C. to about 150° C. and a cooled reactor synthesis effluent 620-1. In some embodiments, heat exchanger 614a can be any of those discussed above for heat exchanger 114a. The reactor synthesis effluent 618 from reactor unit 622 is a heated reactor synthesis effluent having a temperature of from about 250° C. to about 350° C. which is split into first portion 618-1 and second portion 618-2. Accordingly, first portion 618-1 of reactor synthesis effluent 618 delivers the heat in heat exchanger 614a to water feed stream 610 to generate heated water stream 616, and the first portion 618-1 of reactor synthesis effluent 618 is likewise cooled against water feed stream 610 in heat exchanger 614a which cools first portion 618-1 of reactor synthesis effluent 618 to generate cooled reactor synthesis effluent 620-1, i.e., a temperature of less than 120° C. such as from about 80° C. to about 110° C.

System 600 further includes heat exchanger 614b for receiving second portion 618-2 of reactor synthesis effluent 618 from reactor unit 622 and cooling second portion 618-2 of reactor synthesis effluent 618 to generate a cooled reactor synthesis effluent 620-2 having a temperature of from about 80° C. to about 110° C. In some embodiments, heat exchanger 614b can be any of those discussed above for heat exchanger 114b. In this particular embodiment, cooled reactor synthesis effluent 620-1 and cooled reactor synthesis effluent 620-2 are combined into reactor synthesis effluent 620-3 which contains such products as methanol, dimethyl ether, unreacted syngas, etc. The methanol and dimethyl ether products in reactor synthesis effluent 620-3 are separated out from unreacted syngas (also referred to as reactor tail gas) as reactor synthesis effluent 620-4 and sent for further downstream processing as known in the art. For example, reactor synthesis effluent 620-4 can be sent for further downstream processing for creating high value liquid fuels, such as gasoline, diesel, and jet. In some embodiments, heat exchanger 614b can also be used to heat the reactor tail gas 621 before being recycled to the reactor 622.

Reactor tail gas stream 621 which contains mostly untreated syngas separated from reactor synthesis effluent 620 has a temperature of from about 80° C. to about 110° C. and recycled back to reactor unit 622 for further processing to enhance overall energy efficiency.

System 600 further includes reactor unit 622 for receiving heated water stream 616, reactor tail gas 621, and cooled cathode effluent 654-6 as discussed below. In non-limiting illustrative embodiments, reactor unit 622 can be any reactor unit as discussed above for reactor unit 122.

The reaction in reactor unit 622 utilizing at least reactor tail gas 621 and cooled cathode effluent 654-6 (i.e., a cooled syngas effluent) for making products such as, for example, methanol, is an exothermic reaction creating reaction heat to further heat and vaporize the incoming heated water stream 616 to generate a steam feed stream 624 having a temperature of from about 250° C. to about 350° C. In an illustrative embodiment, heated water stream 616 does not participate in the reaction. The reaction conditions and additional components in reactor unit 622 are within the purview of one skilled in the art. The products produced from the reaction process can then be discharged from reactor unit 622 as reactor synthesis effluent 618. Accordingly, for purposes of this illustrative embodiment, reactor synthesis effluent 618 is one or more of methanol and/or dimethyl ether. However, this is merely illustrative and any other chemical product that can be made from the conversion of syngas is contemplated herein for use as reactor synthesis effluent 618.

System 600 further includes heat exchanger 626a for receiving carbon dioxide feed stream 605 and a first portion 654-1 of cathode effluent 654 comprised primarily of syngas, also referred to as heated cathode effluent 654, from the cathode 634-1 of electrolyzer 634 as a heat transfer medium to generate a heated carbon dioxide effluent 628 and a cooled cathode effluent 654-4. In some embodiments, heat exchanger 626a can be any heat exchanger as discussed above for heat exchanger 126a.

The received carbon dioxide feed stream 605 is a gas having a temperature of at or about 20° C. A carbon dioxide source in the present disclosure for carbon dioxide feed stream 605 can be any carbon dioxide source as discussed above.

Referring back to cathode effluent 654, cathode effluent 654 exits the cathode 634-1 of electrolyzer 634 having a temperature of from about 700° C. to about 950° C. or from about 750° C. to about 850° C. and acts as a heat transfer medium in heat exchangers 626a and 626b. When exiting the cathode 634-1 of electrolyzer 634, cathode effluent 654 is split into first portion 654-1, second portion 654-2 and third portion 654-3. Accordingly, first portion 654-1 of cathode effluent 654 delivers the heat in heat exchanger 626a to carbon dioxide feed stream 605 which heats the carbon dioxide feed stream 605 to generate a heated carbon dioxide effluent 628 having a temperature of from about 550° C. to about 650° C., and the first portion 654-1 of cathode effluent 654 is likewise cooled against carbon dioxide feed stream 605 in heat exchanger 626a to generate cooled cathode effluent 654-4, i.e., a temperature of less than 650° C. such as from about 250° C. to about 600° C.

System 600 further includes heat exchanger 626b for receiving a first portion 624-1 of steam feed stream 624 and second portion 654-2 of cathode effluent 654 from the cathode 634-1 of electrolyzer 634 as a heat transfer medium to generate a first heated steam effluent 630-1 and a cooled cathode effluent 654-5. In other words, second portion 654-2 of cathode effluent 654 delivers the heat in heat exchanger 626b to first portion 624-1 of steam feed stream 624 and generates a first heated steam effluent 630-1 having a temperature of from about 550° C. to about 650° C., and the second portion 654-2 of cathode effluent 654 is likewise cooled against first portion 624-1 of steam feed stream 624 in heat exchanger 626b to generate cooled cathode effluent 654-5, i.e., a temperature of less than 650° C. such as from about 250° C. to about 600° C. Cooled cathode effluents 654-4 and 654-5 are then combined as cooled cathode effluent 654-6 and introduced to reactor unit 622 as discussed above. In some embodiments, the cooled cathode effluent 654-6 can be compressed, cooled, and dehydrated before feeding to reactor unit 622. In some embodiments, heat exchanger 526b can be any heat exchanger as discussed above for heat exchanger 126b.

Third portion 654-3 of cathode effluent 654 is recycled back to cathode 634-1 where it is combined with incoming heated carbon dioxide effluent 628, first heated steam effluent 630-1 and either second heated steam effluent 630-2 (FIG. 5A) or heated carbon dioxide and steam effluent 662 (see FIG. 5B) as discussed below.

System 600 further includes heat exchanger 626c for receiving a second portion 624-2 of steam feed stream 624 and a first portion 636-1 of anode effluent 636 from the anode 634-2 of electrolyzer 634 as a heat transfer medium to generate a second heated steam effluent 630-2 and a cooled anode effluent 640, i.e., a temperature of less than 650° C., such as from about 400° C. to about 620° C. As discussed below, anode effluent 636, also referred to as heated anode effluent 636, exits the anode 634-2 of electrolyzer 634 having a temperature of from about 700° C. to about 950° C. or from about 750° C. to about 850° C. and acts as a heat transfer medium in heat exchangers 626c and 632. Anode effluent 636 is split into first portion 636-1 and second portion 636-2. The first portion 636-1 of anode effluent 636 delivers the heat in heat exchanger 626c to heat second portion 624-2 of steam feed stream 624 and provide a second heated steam effluent 630-2 having a temperature of from about 550° C. to about 650° C., and the first portion 636-1 of anode effluent 636 is likewise cooled against second portion 624-2 of steam feed stream 624 in heat exchanger 626c to generate cooled anode effluent 640. In some embodiments, heat exchanger 626c can be any heat exchanger discussed above for heat exchanger 126c.

System 600 further includes heat exchanger 632 for receiving anode purge stream 612 and second portion 636-2 of anode effluent 636 from the anode 634-2 of electrolyzer 634 as a heat transfer medium to generate a heated anode purge stream 644 and a cooled anode effluent 642, i.e., a temperature of less than 650° C. such as from about 400° C. to about 620° C. In non-limiting illustrative embodiments, anode purge stream 612 is one of air, carbon dioxide or an inert gas such as N₂ having a temperature of at or about 20° C. In some embodiments, the anode purge stream 612 may be pressurized as discussed above for anode purge stream 112. In some embodiments, heat exchanger 632 can be any heat exchanger as discussed above for heat exchanger 132.

As discussed above, anode effluent 636 exits the anode 634-2 of electrolyzer 634 having a temperature of from about 700° C. to about 950° C. or from about 750° C. to about 850° C. and acts as a heat transfer medium in heat exchangers 626c and 632. Accordingly, second portion 636-2 of anode effluent 636 delivers the heat in heat exchanger 632 to heat anode purge stream 612 and provide a heated anode purge stream 644 having a temperature of from about 550° C. to about 650° C., and the second portion 636-2 of anode effluent 636 is likewise cooled against anode-forming stream 612 in heat exchanger 632 to generate cooled anode effluent 642.

Heated anode purge stream 644 is then sent to the anode 634-2 of electrolyzer 634 as discussed below. Cooled anode effluents 640 and 642 are then combined as cooled anode effluent 646 and sent for further processing. In an illustrative embodiment, when anode purge stream 612 is air, then cooled anode effluent 646 will be composed primarily of O₂ enrich with air, e.g., about 50% O₂ which can be utilized in a combustion process. As another example, when anode purge stream 612 is carbon dioxide, then cooled anode effluent 646 will be composed of CO₂ and O₂ which can be utilized in an oxy-combustion process.

System 600 further includes electrolyzer 634 for receiving heated carbon dioxide effluent 628, first heated steam effluent 630-1, second heated steam effluent 630-2 and third portion 654-3 of cathode effluent 654 into cathode 634-1 and heated anode purge stream 644 into anode 634-2 where the heated carbon dioxide effluent 628, first heated steam effluent 630-1, second heated steam effluent 630-2 and third portion 654-3 of cathode effluent 654 participate in a reaction to generate corresponding cathode effluent 654 including carbon monoxide (CO) and hydrogen (H₂) and anode effluent 636 including an oxygen enriched stream. Heated anode purge stream 644 participates as a purge gas. In an illustrative embodiment, electrolyzer 634 can be any suitable high temperature electrolyzer as discussed above for electrolyzer 134.

In illustrative embodiments, each of heated carbon dioxide effluent 628, first heated steam effluent 630-1 and second heated steam effluent 630-2 can be further heated to an operating temperature of electrolyzer 634 by supplying additional electricity to electrolyzer 634 so that each of heated carbon dioxide effluent 628, first heated steam effluent 630-1 and second heated steam effluent 630-2 will have a temperature of from about 700° C. to about 950° C., or from about 750° C. to about 850° C. In embodiment, the additional electricity can be received from an intermittent source such as solar power (including photovoltaic and reflective), wind power, tidal power, wave power, batteries, and other intermittent energy sources known in the art and combinations thereof. Alternatively, or in addition, electrolyzer 634 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. The electrolyzer 634 may therefore be electricity connectable to an intermittent energy input, a non-intermittent source, or a combination thereof. In particular embodiments, electrolyzer 634 may receive input energy from the photovoltaic panel.

In an alternative embodiment as illustrated in FIG. 5B, each of heated carbon dioxide effluent 628, first heated steam effluent 630-1 and second heated steam effluent 630-2 can be further heated to an operating temperature of electrolyzer 634 by sending heated carbon dioxide effluent 628, first heated steam effluent 630-1 and second heated steam effluent 630-2 to a heating unit 660 to generate a heated carbon dioxide and steam effluent 662 having a temperature of from about 700° C. to about 950° C., or from about 750° C. to about 850° C. Heating unit 660 can be any conventional heating unit known in the art.

In some embodiment, heating unit 660 may include a heating element, such as a resistive or inductive heating element. The heating unit 660 is configured to heat the heated carbon dioxide effluent 628, first heated steam effluent 630-1 and second heated steam effluent 630-2 to a temperature above the operating temperature of the electrolyzer, i.e., a temperature of from about 700° C. to about 950° C., or from about 750° C. to about 850° C. In another alternate embodiment, the heating unit 660 may include a heat exchanger configured to heat the heated carbon dioxide effluent 628, first heated steam effluent 630-1 and second heated steam effluent 630-2 using heat extracted from a high-temperature fluid, such as a fluid heated to about 1200° C. or more. This fluid may be provided from a solar concentrator farm or a power plant. In some embodiments, the heating unit 660 may include multiple steam heater zones with independent power levels (divided vertically or circumferentially or both), in order to enhance thermal uniformity.

In one or more illustrative embodiments, as depicted in FIGS. 5A and 5B, a system processing environment 700 comprises each of the components of system 600 described herein, as well as a controller 710 operatively coupled to system 600. Controller 710 is configured to control operations of one or more of the components of system 600 discussed above. In one illustrative embodiment, controller 710 can be as described as controller 210.

A system and process for the electrochemical conversion of water and carbon dioxide to produce a mixture of H₂ and CO (i.e., syngas), which is then fed to a Fischer-Tropsch (FT) reactor unit for fuel synthesis utilizing heat sources will now be described with reference to FIGS. 6A and 6B.

Referring now to FIG. 6A, system 800 includes heat exchanger 814a for receiving water feed stream 810 at or around room temperature, i.e., about 20° C., and a first portion 818-1 of Fischer-Tropsch reactor synthesis effluent 818 from Fischer-Tropsch reactor unit 822 as a heat transfer medium to generate a heated water stream 816 having a temperature of about 50° C. to about 150° C. and a cooled Fischer-Tropsch reactor synthesis effluent 820-1. In some embodiments, heat exchanger 814a may be a shell-and-tube, plate-fin, microchannel, spiral wound, or any other suitable heat exchanger. The Fischer-Tropsch reactor synthesis effluent 818 from reactor unit 822 is a Fischer-Tropsch reactor synthesis effluent 818 having a temperature of from about 250° C. to about 350° C. which is split into first portion 818-1 and second portion 818-2. Accordingly, first portion 818-1 of Fischer-Tropsch reactor synthesis effluent 818 delivers the heat in heat exchanger 814a to water feed stream 810 to generate heated water stream 816, and the first portion 818-1 of Fischer-Tropsch reactor synthesis effluent 818 is likewise cooled against water feed stream 810 in heat exchanger 814a which cools first portion 818-1 of Fischer-Tropsch reactor synthesis effluent 818 to generate cooled Fischer-Tropsch reactor synthesis effluent 820-1, i.e., a temperature of less than 300° C. such as from about 200° C. to about 280° C.

System 800 further includes heat exchanger 814b for receiving second portion 818-2 of Fischer-Tropsch reactor synthesis effluent 818 from Fischer-Tropsch reactor unit 822 and cooling second portion 818-2 of Fischer-Tropsch reactor synthesis effluent 818 to generate a cooled Fischer-Tropsch reactor synthesis effluent 820-2 having a temperature of from about 200° C. to about 280° C. In some embodiments, heat exchanger 814b can be any heat exchanger as discussed above for heat exchanger 114b. In this particular embodiment, cooled Fischer-Tropsch reactor synthesis effluent 820-1 and cooled Fischer-Tropsch reactor synthesis effluent 820-2 are combined into Fischer-Tropsch reactor synthesis effluent 820-3 which contains Fischer-Tropsch products as discussed above for Fischer-Tropsch product stream 320-3. In some embodiments, heat exchanger 814b can also be used to heat the reactor tail gas 821-1 before being recycled to the reactor 822.

Accordingly, the Fischer-Tropsch reactor synthesis effluent 818 containing the resulting hydrocarbon product are separated out from the unreacted syngas and byproducts as Fischer-Tropsch reactor synthesis effluent 820-4 and sent for further downstream processing as known in the art. For example, Fischer-Tropsch reactor synthesis effluent 820-4 can be sent for further downstream processing for creating high value liquid fuels, such as gasoline, diesel, and jet. The reactor tail gas 821 composed of unreacted syngas and byproducts separated from Fischer-Tropsch reactor synthesis effluent 820-3 is split into reactor tail gas 821-1 and carbon dioxide enriched tail gas stream 821-2. Reactor tail gas 821-1 is recycled back to the Fischer-Tropsch reactor unit 822 for further Fischer-Tropsch synthesis to increase the overall carbon efficiency. Carbon dioxide enriched tail gas stream 821-2 composed primarily of carbon dioxide and methane is recycled back to the cathode 834-1 of electrolyzer 834 for further syngas generation via electrolysis.

System 800 further includes Fischer-Tropsch reactor unit 822 for heating heated water stream 816 via a Fischer-Tropsch reaction of reactor tail gas 821-1, and cooled cathode effluent 854-6 for Fischer-Tropsch synthesis as discussed below. In non-limiting illustrative embodiments, Fischer-Tropsch reactor unit 822 can be the same as discussed above for Fischer-Tropsch reactor 322.

The Fischer-Tropsch synthesis utilizing reactor tail gas 821-1 and cooled cathode effluent 854-6 is an exothermic reaction creating reaction heat to further heat and vaporize the incoming heated water stream 816 to generate a steam feed stream 824 having a temperature of from about 250° C. to about 350° C. The hydrocarbon products produced from the Fischer-Tropsch process are then discharged from reactor unit 822 as Fischer-Tropsch reactor synthesis effluent 818.

System 800 further includes heat exchanger 826a for receiving carbon dioxide feed stream 805 and a first portion 854-1 of cathode effluent 854 from the cathode 834-1 of electrolyzer 834 as a heat transfer medium to generate a heated carbon dioxide effluent 828 and a cooled cathode effluent 854-4. In some embodiments, heat exchanger 826a can be any heat exchanger as discussed above for heat exchanger 126a. The received carbon dioxide feed stream 805 is a gas having a temperature of at or about 20° C. A carbon dioxide source in the present disclosure for carbon dioxide feed stream 805 can be obtained from any of the sources discussed above for carbon dioxide feed stream 105.

Referring back to cathode effluent 854, cathode effluent 854, also referred to as cathode effluent 854, exits the cathode 834-1 of electrolyzer 834 having a temperature of from about 700° C. to about 950° C. or from about 750° C. to about 850° C. and acts as a heat transfer medium in heat exchangers 826a and 826b. Cathode effluent 854 from the cathode 834-1 of electrolyzer 834 is split into first portion 854-1, second portion 854-2 and third portion 854-3. Accordingly, first portion 854-1 of cathode effluent 854 delivers the heat in heat exchanger 826a to carbon dioxide feed stream 805 which heats the carbon dioxide feed stream 805 to generate a heated carbon dioxide effluent 828 having a temperature of from about 550° C. to about 650° C., and the first portion 854-1 of cathode effluent 854 is likewise cooled against carbon dioxide feed stream 805 in heat exchanger 826a to generate a cooled cathode effluent 854-4, i.e., a temperature of less than 650° C. such as from about 250° C. to about 600° C. In some embodiments, heat exchanger 826a can be any of those discussed above for heat exchanger 126a.

System 800 further includes heat exchanger 826b for receiving a first portion 824-1 of steam feed stream 824 and second portion 854-2 of cathode effluent 854 from the cathode 834-1 of electrolyzer 834 as a heat transfer medium to generate a first heated steam effluent 830-1 and a cooled cathode effluent 854-5. In other words, second portion 854-2 of cathode effluent 854 delivers the heat in heat exchanger 826b to heat first portion 824-1 of steam feed stream 824 and generate a first heated steam effluent 830-1 having a temperature of from about 550° C. to about 650° C., and the second portion 854-2 of cathode effluent 854 is likewise cooled against first portion 824-1 of steam feed stream 824 in heat exchanger 826b to generate a cooled cathode effluent 854-5, i.e., a temperature of less than 650° C. such as from about 250° C. to about 600° C. Cooled cathode effluents 854-4 and 854-5 are then combined as cooled cathode effluent 854-6 and introduced to Fischer-Tropsch reactor unit 822 for further processing as discussed above. In some embodiments, the cooled cathode effluent 854-6 can be compressed, cooled, and dehydrated before feeding to Fischer-Tropsch reactor unit 822. In some embodiments, heat exchanger 826b can be any of those discussed above for heat exchanger 126b.

Third portion 854-3 of cathode effluent 854 is recycled back to cathode 834-1 where it is combined with incoming heated carbon dioxide effluent 828, first heated steam effluent 830-1, second heated steam effluent 830-2 and carbon dioxide enriched tail gas stream 821-2 (FIG. 6A) or heated carbon dioxide and steam effluent 862 (see FIG. 6B) as discussed below.

System 800 further includes heat exchanger 826c for receiving a second portion 824-2 of steam feed stream 824 and a first portion 836-1 of anode effluent 836, i.e., an oxygen enriched stream, from the anode 834-2 of electrolyzer 834 as a heat transfer medium to generate a second heated steam effluent 830-2 and a cooled anode effluent 840. In other words, anode effluent 836, also referred to as heated anode effluent 836, exits the anode 834-2 of electrolyzer 834 having a temperature of from about 700° C. to about 950° C. or from about 750° C. to about 850° C. and acts as a heat transfer medium in heat exchangers 826c and 832. Anode effluent 836 is split into first portion 836-1 and second portion 836-2. Accordingly, first portion 836-1 of anode effluent 836 delivers the heat in heat exchanger 826c to heat second portion 824-2 of steam feed stream 824 and provide a second heated steam effluent 830-2 having a temperature of from about 550° C. to about 650° C., and the first portion 836-1 of anode effluent 836 is likewise cooled against second portion 824-2 of steam feed stream 824 in heat exchanger 826c to generate cooled anode effluent 840, i.e., a temperature of less than 650° C. such as from about 400° C. to about 620° C. In some embodiments, heat exchanger 826c can be any heat exchanger as discussed above for heat exchanger 126c.

System 800 further includes heat exchanger 832 for receiving anode purge stream 812 and second portion 836-2 of anode effluent 836 from the anode 834-2 of electrolyzer 834 as a heat transfer medium to generate a heated anode purge stream 844 and a cooled anode effluent 842. The received anode purge stream 812 is one of air, carbon dioxide or an inert gas such as N₂ having a temperature of at or about 20° C. In some embodiments, the anode purge stream 812 may be pressurized as discussed above for anode purge stream 112. In some embodiments, heat exchanger 832 can be any of those discussed above for heat exchanger 132.

As discussed above, anode effluent 836 exits the anode 834-2 of electrolyzer 834 having a temperature of from about 700° C. to about 950° C. or from about 750° C. to about 850° C. and acts as a heat transfer medium in heat exchangers 826c and 832. Accordingly, second portion 836-2 of anode effluent 836 delivers the heat in heat exchanger 832 to heat anode purge stream 812 and provide a heated anode purge stream 844 having a temperature of from about 550° C. to about 650° C., and the second portion 836-2 of anode effluent 836 is likewise cooled against anode purge stream 812 in heat exchanger 832 to generate cooled anode effluent 842, i.e., a temperature of less than 650° C. such as from about 400° C. to about 620° C. Heated anode purge stream 844 is then sent to the anode 834-2 of electrolyzer 834 as discussed below.

Cooled anode effluents 840 and 842 are then combined as cooled anode effluent 846 and sent for further processing. In an illustrative embodiment, when anode purge stream 812 is air, then cooled anode effluent 846 will be composed primarily of O₂ enrich with air, e.g., about 50% O₂ which can be utilized in a combustion process. As another example, when anode purge stream 812 is carbon dioxide, then cooled anode effluent 846 will be composed of CO₂ and O₂ which can be utilized in an oxy-combustion process.

System 800 further includes electrolyzer 834 for receiving heated carbon dioxide effluent 828, first heated steam effluent 830-1, second heated steam effluent 830-2, third portion 854-3 of cathode effluent 854 and carbon dioxide enriched tail gas stream 821-2 into cathode 834-1 and heated anode purge stream 844 into anode 834-2 where the heated carbon dioxide effluent 828, first heated steam effluent 830-1, second heated steam effluent 830-2, third portion 854-3 of cathode effluent 854 and carbon dioxide enriched tail gas stream 821-2 participate in a reaction to generate corresponding cathode effluent 854 including syngas composed of carbon monoxide (CO) and hydrogen (H₂) and anode effluent 836 including an oxygen enriched stream, and heated anode purge stream 844 participates as a purge gas. The electrolyzer 834 can be any suitable high temperature electrolyzer as discussed above for electrolyzer 134.

In illustrative embodiments, each of heated carbon dioxide effluent 828, first heated steam effluent 830-1 and second heated steam effluent 830-2 can be further heated to an operating temperature of electrolyzer 834 by supplying additional electricity to electrolyzer 834 so that each of heated carbon dioxide effluent 828, first heated steam effluent 830-1 and second heated steam effluent 830-2 will have a temperature of from about 700° C. to about 950° C., or from about 750° C. to about 850° C. In embodiment, the additional electricity can be as discussed above for electrolyzer 634.

In an alternative embodiment as illustrated in FIG. 6B, each of heated carbon dioxide effluent 828, first heated steam effluent 830-1, second heated steam effluent 830-2 and carbon dioxide enriched tail gas stream 821-2 can be further heated to an operating temperature of electrolyzer 834 by sending heated carbon dioxide effluent 828, first heated steam effluent 830-1, second heated steam effluent 830-2 and carbon dioxide enriched tail gas stream 821-2 to a heating unit 860 to generate a heated carbon dioxide and steam effluent 862 having a temperature of from about 700° C. to about 950° C., or from about 750° C. to about 850° C. Heating unit 860 can be the same as discussed above for heating unit 660.

In one or more illustrative embodiments, as depicted in FIGS. 6A and 6B, a system processing environment 900 comprises each of the components of system 800 described herein, as well as a controller 910 operatively coupled to system processing environment 900. Controller 910 is configured to control operations of one or more of the components of system 800 discussed above. In one illustrative embodiment, controller 910 is configured to actuate one or more of the functionalities of system 800 described herein. For example, controller 910 can be similar to controller 210 described above and function in a similar manner.

According to an aspect of the present disclosure, a method comprises:
heating a carbon dioxide feed stream in a first heat exchanger using a first cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
heating a first steam feed stream having a temperature of about 250° C. to about 350° C. in a second heat exchanger using a second cathode effluent from the cathode of the electrolyzer as a heat transfer medium to generate a first heated steam effluent having a temperature of about 550° C. to about 650° C.,
heating a second steam feed stream having a temperature of about 250° C. to about 350° C. in a third heat exchanger using an anode effluent from the anode of the electrolyzer as a heat transfer medium to generate a second heated steam effluent having a temperature of about 550° C. to about 650° C.,
combusting, in a combustion unit, a tail gas stream to transfer heat from the combusting to the heated carbon dioxide effluent, the first heated steam effluent and the second heated steam effluent to generate a heated carbon dioxide and steam stream effluent having a temperature of about 700° C. to about 950° C.; and
passing the heated carbon dioxide and steam stream effluent to the cathode of the electrolyzer.

In one or more additional illustrative embodiments, as may be combined with the preceding paragraphs, the heated carbon dioxide and steam stream effluent has a temperature of about 750° C. to about 850° C.

In one or more additional illustrative embodiments, as may be combined with the preceding paragraphs, where heating the first steam feed stream further comprises generating a first cooled cathode effluent and heating the carbon dioxide feed stream further comprises generating a second cooled cathode effluent.

In one or more additional illustrative embodiments, as may be combined with the preceding paragraphs, the method further comprising heating a water feed stream in a third heat exchanger using a reactor synthesis effluent from a reactor unit as a heat transfer medium to generate a heated water effluent having a temperature of about 50° C. to about 150° C., introducing the heated water effluent, the first cooled cathode effluent and the second cooled cathode effluent to the reactor unit, performing an exothermic reaction in the reactor unit, thereby transferring heat from the exothermic reaction to the heated water effluent to vaporize the heated water effluent and generate a third steam feed stream having temperature of about 250° C. to about 350° C., and splitting the third steam feed stream into the first steam feed stream and the second steam feed stream.

In one or more additional illustrative embodiments, as may be combined with the preceding paragraphs, where the first cooled cathode effluent and the second cooled cathode effluent are combined into a third cooled cathode effluent, and the method further comprises passing the third cooled cathode effluent to the reactor unit, wherein the first cooled cathode effluent and the second cooled cathode effluent are each a first cooled syngas effluent and a second cooled syngas effluent.

In one or more additional illustrative embodiments, as may be combined with the preceding paragraphs, where the exothermic reaction in the reactor unit produces a chemical product or a fuel.

In one or more additional illustrative embodiments, as may be combined with the preceding paragraphs, where the chemical product is one or more of methanol and dimethyl ether and the fuel is one or more of gasoline, diesel, and jet fuel.

In one or more additional illustrative embodiments, as may be combined with the preceding paragraphs, where the exothermic reaction in the reactor unit produces a Fischer-Tropsch product.

In one or more additional illustrative embodiments, as may be combined with the preceding paragraphs, where the exothermic reaction in the reactor unit comprises converting a stream comprising the first cooled syngas effluent and the second cooled syngas effluent to the reactor synthesis effluent including tail gas.

In one or more additional illustrative embodiments, as may be combined with the preceding paragraphs, where the method further comprises splitting the reactor synthesis effluent into a first reactor synthesis effluent and a second reactor synthesis effluent, heating the water feed stream in the third heat exchanger using the first reactor synthesis effluent as a heat transfer medium to generate the heated water effluent and a cooled first reactor synthesis effluent, cooling the second reactor synthesis effluent in a fourth heat exchanger to generate a cooled second reactor synthesis effluent, combining the cooled first reactor synthesis effluent and the cooled second reactor synthesis effluent to form a third cooled reactor synthesis effluent, separating the tail gas from the third cooled reactor synthesis effluent into a first tail gas stream and a second tail gas stream, and passing the first tail gas stream to the combustion unit for combustion.

In one or more additional illustrative embodiments, as may be combined with the preceding paragraphs, the method further comprising heating an anode purge stream in a third heat exchanger using another anode effluent from the anode of the electrolyzer as a heat transfer medium to generate a heated anode purge stream having a temperature of about 550° C. to about 650° C., and heating the heated anode purge stream to about 750° C. to about 850° C. utilizing a combustion effluent from the combustion unit.

In one or more additional illustrative embodiments, as may be combined with the preceding paragraphs, the method further comprising compressing the anode purge stream to a pressure of about 1 bar to about 2 bar prior to heating in the third heat exchanger.

In one or more additional illustrative embodiments, as may be combined with the preceding paragraphs, where the electrolyzer is a solid oxide electrolyzer.

According to an aspect of the present disclosure, a method comprises:
heating a water feed stream in a first heat exchanger using a reactor synthesis effluent including tail gas from a reactor unit as a heat transfer medium to generate a heated water effluent having a temperature of about 50° C. to about 150° C. and a cooled reactor synthesis effluent including the tail gas,
separating the tail gas from the cooled reactor synthesis effluent to generate a first tail gas stream and a second tail gas stream,
introducing the first tail gas stream, a cooled cathode effluent and the heated water effluent to the reactor unit,
performing an exothermic reaction comprising the first tail gas stream and the cooled cathode effluent in the reactor unit thereby transferring heat from the exothermic reaction to the heated water effluent to generate a steam feed stream having a temperature of about 250° C. to about 350° C.,
splitting the steam feed stream to a first steam feed stream and a second steam feed stream,
heating a carbon dioxide feed stream in a second heat exchanger using a first heated cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
heating the first steam feed stream in a third heat exchanger using a second heated syngas effluent from the cathode of the electrolyzer as a heat transfer medium to generate a first heated steam effluent having a temperature of about 550° C. to about 650° C.,
heating the second steam feed stream in a fourth heat exchanger using an anode effluent from the anode of the electrolyzer as a heat transfer medium to generate a second heated steam effluent having a temperature of about 550° C. to about 650° C.,
combusting, in a combustion unit, the second tail gas stream to transfer heat from the combusting to heat the heated carbon dioxide effluent, the first heated steam effluent and the second heated steam effluent and generate a heated carbon dioxide and steam stream effluent having a temperature of about 700° C. to about 950° C., and
passing the heated carbon dioxide and steam stream effluent to the cathode of the electrolyzer.

In one or more additional illustrative embodiments, as may be combined with the preceding paragraphs, where the exothermic reaction in the reactor unit comprises converting the cooled cathode effluent and the first tail gas stream to a chemical product or a fuel.

In one or more additional illustrative embodiments, as may be combined with the preceding paragraphs, where the chemical product is one or more of methanol and dimethyl ether and the fuel is one or more of gasoline, diesel, and jet fuel.

In one or more additional illustrative embodiments, as may be combined with the preceding paragraphs, where the exothermic reaction in the reactor unit comprises converting the cooled cathode effluent and the first tail gas stream to a Fischer-Tropsch product.

In one or more additional illustrative embodiments, as may be combined with the preceding paragraphs, the method further comprising heating an anode purge stream in a fifth heat exchanger using another anode effluent from the anode of the electrolyzer as a heat transfer medium to generate a heated anode purge stream having a temperature of about 550° C. to about 650° C., and heating the heated anode purge stream to about 750° C. to about 850° C. utilizing a combustion effluent from the combustion unit.

In one or more additional illustrative embodiments, as may be combined with the preceding paragraphs, where the electrolyzer is a solid oxide electrolyzer.

According to an aspect of the present disclosure, a system comprises:
a first heat exchanger configured to heat a carbon dioxide feed stream using a first cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.,
a second heat exchanger configured to heat a first steam feed stream having a temperature of about 250° C. to about 350° C. using a second cathode effluent from the cathode of the electrolyzer as a heat transfer medium to generate a first heated steam effluent having a temperature of about 550° C. to about 650° C.,
a third heat exchanger configured to heat a second steam feed stream having a temperature of about 250° C. to about 350° C. using an anode effluent from the anode of the electrolyzer as a heat transfer medium to generate a second heated steam effluent having a temperature of about 550° C. to about 650° C., and
a combustion unit configured to combust a tail gas feed stream to transfer heat from the combusting to the heated carbon dioxide effluent, the first heated steam effluent and the second heated steam effluent to generate a heated carbon dioxide and steam stream effluent having a temperature of about 700° C. to about 950° C. for sending to the cathode of the electrolyzer.

In one or more additional illustrative embodiments, as may be combined with the preceding paragraphs, the system further comprises:
a reactor unit configured to perform an exothermic reaction with a tail gas stream and a cooled cathode effluent thereby transferring heat from the exothermic reaction to a heated water effluent to generate the first and second steam feed streams having a temperature of about 250° C. to about 350° C.

In one or more additional illustrative embodiments, as may be combined with the preceding paragraphs, the reactor unit is further configured to perform the exothermic reaction of the tail gas stream and the cooled cathode effluent to produce a chemical product or a fuel.

In one or more additional illustrative embodiments, as may be combined with the preceding paragraphs, the chemical product is one or more of methanol and dimethyl ether and the fuel is one or more of gasoline, diesel, and jet fuel.

In one or more additional illustrative embodiments, as may be combined with the preceding paragraphs, the reactor unit is further configured to perform the exothermic reaction of the tail gas stream and the cooled cathode effluent to produce a Fischer-Tropsch product.

In one or more additional illustrative embodiments, as may be combined with the preceding paragraphs, the system further comprises:
a fourth heat exchanger configured to heat a water feed stream using a reactor synthesis effluent including tail gas from the reactor unit as a heat transfer medium to generate the heated water effluent having a temperature of about 50° C. to about 150° C. and a cooled reactor synthesis effluent including the tail gas.

In one or more additional illustrative embodiments, as may be combined with the preceding paragraphs, the system further comprises:
a fifth heat exchanger configured to heat an anode purge stream using another anode effluent from the anode of the electrolyzer as a heat transfer medium to generate a heated anode purge stream having a temperature of about 550° C. to about 650° C.

In one or more additional illustrative embodiments, as may be combined with the preceding paragraphs, the system further comprises:
a sixth heat exchanger configured to heat the heated anode purge stream using a combustion effluent from the combustion unit as a heat transfer medium to generate another heated anode purge stream having a temperature of about 700° C. to about 950° C. for sending to the anode of the electrolyzer.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A method, comprising:
heating a carbon dioxide feed stream in a first heat exchanger using a first cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.;
heating a first steam feed stream having a temperature of about 250° C. to about 350° C. in a second heat exchanger using a second cathode effluent from the cathode of the electrolyzer as a heat transfer medium to generate a first heated steam effluent having a temperature of about 550° C. to about 650° C.;
heating a second steam feed stream having a temperature of about 250° C. to about 350° C. in a third heat exchanger using an anode effluent from the anode of the electrolyzer as a heat transfer medium to generate a second heated steam effluent having a temperature of about 550° C. to about 650° C.;
combusting, in a combustion unit, a tail gas stream to transfer heat from the combusting to the heated carbon dioxide effluent, the first heated steam effluent and the second heated steam effluent to generate a heated carbon dioxide and steam stream effluent having a temperature of about 700° C. to about 950° C.; and
passing the heated carbon dioxide and steam stream effluent to the cathode of the electrolyzer.

2. The method according to claim 1, wherein the heated carbon dioxide and steam stream effluent has a temperature of about 750° C. to about 850° C.

3. The method according to claim 1, wherein heating the carbon dioxide feed stream further comprises generating a first cooled cathode effluent and heating the first steam feed stream further comprises generating a second cooled cathode effluent.

4. The method according to claim 3, further comprising:
heating a water feed stream in a third heat exchanger using a reactor synthesis effluent from a reactor unit as a heat transfer medium to generate a heated water effluent having a temperature of about 50° C. to about 150° C.;
introducing the heated water effluent, the first cooled cathode effluent and the second cooled cathode effluent to the reactor unit;
performing an exothermic reaction in the reactor unit, thereby transferring heat from the exothermic reaction to the heated water effluent to vaporize the heated water effluent and generate a third steam feed stream having temperature of about 250° C. to about 350° C.; and
splitting the third steam feed stream into the first steam feed stream and the second steam feed stream.

5. The method according to claim 4, wherein the first cooled cathode effluent and the second cooled cathode effluent are combined into a third cooled cathode effluent, and the method further comprises passing the third cooled cathode effluent to the reactor unit, wherein the first cooled cathode effluent and the second cooled cathode effluent are each a first cooled syngas effluent and a second cooled syngas effluent.

6. The method according to claim 5, wherein the exothermic reaction in the reactor unit produces a chemical product or a fuel.

7. The method according to claim 6, wherein the chemical product is one or more of methanol and dimethyl ether and the fuel is one or more of gasoline, diesel, and jet fuel.

8. The method according to claim 5, wherein the exothermic reaction in the reactor unit produces a Fischer-Tropsch product.

9. The method according to claim 5, wherein the exothermic reaction in the reactor unit comprises converting streams comprising the first cooled syngas effluent and the second cooled syngas effluent to the reactor synthesis effluent including tail gas, and the method further comprises:
splitting the reactor synthesis effluent into a first reactor synthesis effluent and a second reactor synthesis effluent;
heating the water feed stream in the third heat exchanger using the first reactor synthesis effluent as a heat transfer medium to generate the heated water effluent and a cooled first reactor synthesis effluent;
cooling the second reactor synthesis effluent in a fourth heat exchanger to generate a cooled second reactor synthesis effluent;
combining the cooled first reactor synthesis effluent and the cooled second reactor synthesis effluent to form a third cooled reactor synthesis effluent;
separating the tail gas from the third cooled reactor synthesis effluent into a first tail gas stream and a second tail gas stream; and
passing the first tail gas stream to the combustion unit for combustion.

10. The method according to claim 1, further comprising:
heating an anode purge stream in a third heat exchanger using another anode effluent from the anode of the electrolyzer as a heat transfer medium to generate a heated anode purge stream having a temperature of about 550° C. to about 650° C.; and
heating the heated anode purge stream to about 750° C. to about 850° C. utilizing a combustion effluent from the combustion unit.

11. The method according to claim 10, further comprising compressing the anode purge stream to a pressure of about 1 bar to about 2 bar prior to heating in the third heat exchanger.

12. The method according to claim 1, wherein the electrolyzer is a solid oxide electrolyzer.

13. A method, comprising:
heating a water feed stream in a first heat exchanger using a reactor synthesis effluent including tail gas from a reactor unit as a heat transfer medium to generate a heated water effluent having a temperature of about 50° C. to about 150° C. and a cooled reactor synthesis effluent including the tail gas;
separating the tail gas from the cooled reactor synthesis effluent to generate a first tail gas stream and a second tail gas stream;
introducing the first tail gas stream, a cooled cathode effluent and the heated water effluent to the reactor unit;
performing an exothermic reaction comprising the first tail gas stream and the cooled cathode effluent in the reactor unit thereby transferring heat from the exothermic reaction to the heated water effluent to generate a steam feed stream having a temperature of about 250° C. to about 350° C.;
splitting the steam feed stream to a first steam feed stream and a second steam feed stream;
heating a carbon dioxide feed stream in a second heat exchanger using a first heated syngas effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.;
heating the first steam feed stream in a third heat exchanger using a second heated syngas effluent from the cathode of the electrolyzer as a heat transfer medium to generate a first heated steam effluent having a temperature of about 550° C. to about 650° C.;
heating the second steam feed stream in a fourth heat exchanger using an anode effluent from the anode of the electrolyzer as a heat transfer medium to generate a second heated steam effluent having a temperature of about 550° C. to about 650° C.;
combusting, in a combustion unit, the second tail gas stream to transfer heat from the combusting to heat the heated carbon dioxide effluent, the first heated steam effluent and the second heated steam effluent and generate a heated carbon dioxide and steam stream effluent having a temperature of about 700° C. to about 950° C.; and
passing the heated carbon dioxide and steam stream effluent to the cathode of the electrolyzer.

14. The method according to claim 13, wherein the exothermic reaction in the reactor unit comprises converting the cooled cathode effluent and the first tail gas stream to a chemical product or a fuel.

15. The method according to claim 14, wherein the chemical product is one or more of methanol and dimethyl ether and the fuel is one or more of gasoline, diesel, and jet fuel.

16. The method according to claim 13, wherein the exothermic reaction in the reactor unit comprises converting the cooled cathode effluent and the first tail gas stream to a Fischer-Tropsch product.

17. The method according to claim 13, further comprising:
heating an anode purge stream in a fifth heat exchanger using another anode effluent from the anode of the electrolyzer as a heat transfer medium to generate a heated anode purge stream having a temperature of about 550° C. to about 650° C., and
heating the heated anode purge stream to about 750° C. to about 850° C. using a combustion effluent from the combustion unit.

18. The method according to claim 13, wherein the electrolyzer is a solid oxide electrolyzer.

19. A system, comprising:
a first heat exchanger configured to heat a carbon dioxide feed stream using a first cathode effluent from a cathode of an electrolyzer comprising an anode, a cathode, and an electrolyte inserted between the anode and the cathode as a heat transfer medium to generate a heated carbon dioxide effluent having a temperature of about 550° C. to about 650° C.;
a second heat exchanger configured to heat a first steam feed stream having a temperature of about 250° C. to about 350° C. using a second cathode effluent from the cathode of the electrolyzer as a heat transfer medium to generate a first heated steam effluent having a temperature of about 550° C. to about 650° C.;
a third heat exchanger configured to heat a second steam feed stream having a temperature of about 250° C. to about 350° C. using an anode effluent from the anode of the electrolyzer as a heat transfer medium to generate a second heated steam effluent having a temperature of about 550° C. to about 650° C.; and
a combustion unit configured to combust a tail gas feed stream to transfer heat from the combusting to the heated carbon dioxide effluent, the first heated steam effluent and the second heated steam effluent to generate a heated carbon dioxide and steam stream effluent having a temperature of about 700° C. to about 950° C. for sending to the cathode of the electrolyzer.

20. The system according to claim 19, further comprising:
a reactor unit configured to perform an exothermic reaction with a tail gas stream and a cooled cathode effluent thereby transferring heat from the exothermic reaction to a heated water effluent to generate the first steam feed stream and the second steam feed stream each having a temperature of about 250° C. to about 350° C.
